(19) United States
(12) Patent Application Publication
(10) Pub. No.: US 2025/0063947 A1
(43) Pub. Date: Feb. 20, 2025

(54) COMPOUND AND ITS USE IN ORGANIC ELECTRONIC DEVICES

(71) Applicant: Heliatek GmbH, Dresden (DE)

(72) Inventors: Dirk Hildebrandt; Olga Gerdes; Nina Grimm

(21) Appl. No.: 18/724,198

(22) PCT Filed: Dec. 29, 2022
(86) PCT No.: PCT/EP2022/088022

§ 371 Date: Jun. 26, 2024

(30) Foreign Application Priority Data
Dec. 30, 2021 (EP) 21218393.3

(51) Int. Cl. H10K 85/60; C07D 495/04; C07D 519/00; H10K 30/40

(52) U.S. Cl. CPC H10K 85/6572; C07D 495/04; C07D 519/00; H10K 85/654; H10K 85/657; H10K 85/6574; H10K 85/6576; H10K 30/40

(57) ABSTRACT

The invention relates to a compound of the general formula (I), a use of such a compound in an organic electronic device, and an organic electronic device comprising such a compound.

The invention relates to a compound of the general formula (I), a use of such a compound in an organic electronic device, and an organic electronic device comprising such a compound.

Electronic devices made of organic materials are known for use as LED's (OLED) and organic photovoltaic elements (OPV), i.e., organic solar cells. The organic materials used fulfill different functions in these organic electronic devices—in particular, charge transport, light emission, or light absorption. Organic materials in opto-electronic devices can be polymers or small molecules and can be processed in solution or emulsion by means of wet-chemical processes, such as coating or printing, or in a vacuum by sublimation to form thin layers. Organic electronic devices are, for example, displays, data memories, or transistors, but also organic optoelectronic devices, in particular, solar cells or photodetectors. Solar cells or photodetectors have a photoactive layer in which electron-hole pairs (excitons) bonded when electromagnetic radiation is applied are produced as charge carriers. The excitons pass by diffusion to an interface at which electrons and holes are separated from one another. The material which absorbs the electrons is referred to as an acceptor, and the material which absorbs the holes is referred to as a donor. Further organic electronic devices are light-emitting devices which emit light when current is passed through them. Organic electronic devices comprise at least two electrodes, wherein one electrode is applied to a substrate, and the other functions as a counter-electrode. Between the electrodes there is at least one photoactive layer-preferably an organic photoactive layer. Further layers, e.g., transport layers, can be arranged between the electrodes.

As above, organic semi-conductive materials are sought which, when used in organic electronic devices, lead to an improvement in the properties of the devices. The absorption spectrum of known absorbers does not completely cover the blue spectral range. For increasing the absorption range and thus for increasing the efficiency of solar cells, absorbers are therefore required which absorb strongly in the spectral range between 400 nm and 600 nm, and in particular between 480 and 580 nm, and have a voltage in the range of 1 V. This is particularly advantageous for use in tandem and triplet cells.

Organic compounds for electrical devices are known from the prior art. US20190043926A1 discloses organic compounds for photoelectric transducer elements for converting electricity into radiation. Such compounds are not stable when vaporized in a vacuum and can therefore only be processed from liquid.

A particular disadvantage of the prior art is that the voltage of many absorber materials in an absorption range is too low, in particular in the range of well below 1.0 V, especially in the range of 0.9 V to 0.95 V. With multiple cells, however, the cell with the lowest voltage is the limiting factor for the entire electrical device.

The object is achieved by the subject matter of the independent claims. Advantageous embodiments can be found in the dependent claims.

The object is achieved in particular by the provision of a compound of the general formula I

A1-M-(T1)ₐ-(T2)_{b}-(T3)_{c}-A2 (I)

with the parameters a, b, c in each case independently of one another 0, 1 or 2, with the proviso that at least one of the parameters a, b, c=1 or 2, with group M selected from the group consisting of:

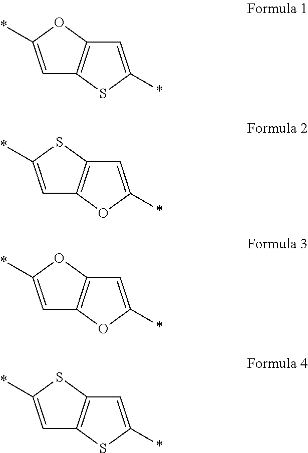 [SMILES: *c1cc2sc(*)cc2o1 ; *c1cc2sc(*)cc2o1 ; *c1cc2oc(*)cc2o1 ; *c1cc2sc(*)cc2s1]

wherein * denotes the link to the groups T1, T2, T3, A1 and A2, wherein H atoms of Formulas 1 to 4 may be substituted by alkyl, O-alkyl, S-alkyl or halogen;
with the groups T1, T2 and T3 independently selected from the group consisting of:

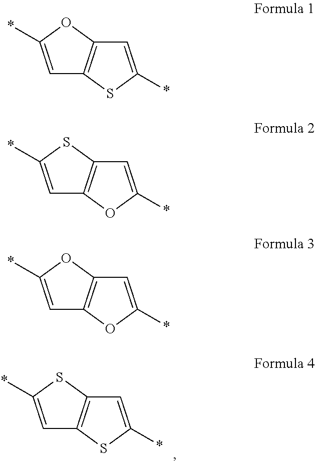 [SMILES: *c1cc2sc(*)cc2o1 ; *c1cc2sc(*)cc2o1 ; *c1cc2oc(*)cc2o1 ; *c1cc2sc(*)cc2s1]

wherein H atoms of Formulas 1 to 4 may be substituted by alkyl, O-alkyl, S-alkyl or halogen,

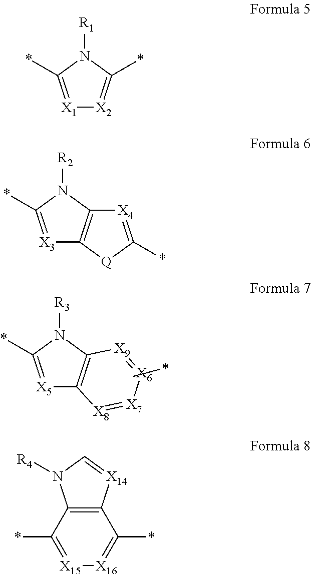

with R1 to R4 each independently selected from the group consisting of H, alkyl, alkenyl, aryl and heteroaryl;

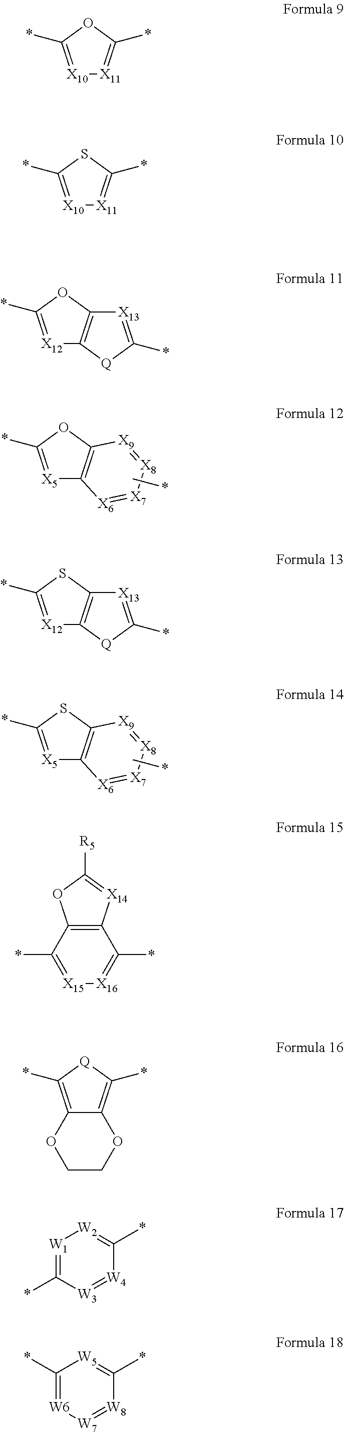

with X₁ to X₁₆, selected independently from N or CR₆, with the proviso that, in the Formulas 7, 12 and 14, a group of X₆ to X₉ is denoted by the link * to a further group in the compound of the general Formula I, with Q selected from the group consisting of O, S, and NR₇, with W₁ to W₈ each independently selected from N and CR₈, wherein R₆, R₇ and R₈ are each independently selected from the group consisting of H, CN, alkyl, alkenyl, O-alkyl, S-alkyl, aryl, and heteroaryl, wherein H atoms may be substituted by alkyl, alkenyl, O-alkyl, S-alkyl, aryl, and heteroaryl;
wherein the electron-attracting groups A1 and A2 are, independently of one another, electron-attracting groups having at least one C═C double bond, and wherein * denotes the link to a further group M, T1, T2, T3 and A2 in the compound of the general formula I.

The conjugated TT-electron system of the donor region (M-(T1)ₐ-(T2)_{b}-(T3)_{c}) of the compounds of the general formula I can be extended beyond the donor block M with the electron-attracting groups A1 and A2 by incorporating at least one further donor block T1, T2 and/or T3. According to the invention, in particular the building block with a fused furan-thiophene, thiophene-furan, furan-furan, and/or thiophene-thiophene building block:

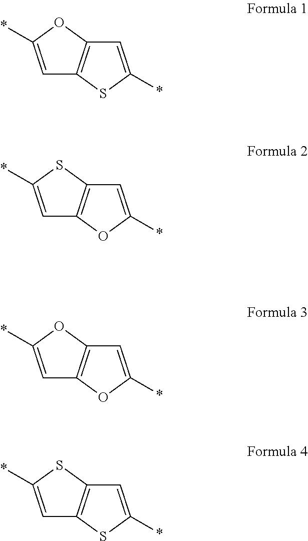 [SMILES: *c1cc2sc(*)cc2o1 ; *c1cc2sc(*)cc2o1 ; *c1cc2oc(*)cc2o1 ; *c1cc2sc(*)cc2s1]

is important, especially to increase the aromaticity of the compounds. In particular, the component leads to a lowering of the HOMO within the compound, as a result of which the compound supplies more voltage in an electronic device. Due to this structural feature of group M, the present compounds have a particularly high optical density, preferably in the visible spectral range, and in particular a high integral over the optical density in the absorption spectrum compared to compounds not according to the invention which do not have the structural element described above. Here, an “integral” is understood to mean the area content below a curve in the absorption spectrum, which is an important feature for the suitability of the material as photosensitive material.

According to the present invention, a heteroatom is understood to mean in particular O, S or N.

For the purposes of the present invention, substituted or a substituent means in particular the exchange of one or more H atoms for another group or another atom. A substituent is in particular a halogen or a pseudohalogen, preferably F, Cl or CN, an alkyl group, an alkenyl group or an aryl group.

In a particularly preferred embodiment of the invention, b=0.

In a particularly preferred embodiment of the invention, T1 and T3 are each independently selected from Formulas 1 to 4.

The chemical compounds according to the invention of the general formula I have advantages compared to the prior art. Advantageously, improved absorbers for organic electronic devices, and in particular photovoltaic elements, can be provided. Advantageously, absorber materials are provided, for the red and near-infrared spectral range, with a high absorption strength. Advantageously, the absorbers have an increased photovoltage compared to other ADA absorbers without a fused-on furan-thiophene, thiophene-furan, furan-furan, and/or thiophene-thiophene building block (Formula 1 to 4). Advantageously, the open-circuit voltage Uoc of electronic device with a compound according to the invention is increased. Advantageously, the compounds according to the invention absorb particularly strongly in the spectral range between 400 nm and 600 nm, in particular between 480 and 580 nm, and supply a voltage in an electronic device in the range of 1 V. Advantageously, the efficiency of photovoltaic elements can be increased. Advantageously, the compounds according to the invention can be evaporated largely without residue in a vacuum, and are thus suitable for vacuum processing for producing photovoltaic elements. This is particularly advantageous in the case of multiple cells, and preferably tandem or triplet cells, or mixed layers, since the smallest voltage of the layer is limiting for the total voltage of the cell.

The compounds according to the invention are in particular so-called “small molecules,” wherein this means non-polymeric oligomeric organic molecules having a molar mass of between 100 and 2000 g/mol.

According to a development of the invention, it is provided that T1 is independently selected from the group consisting of:

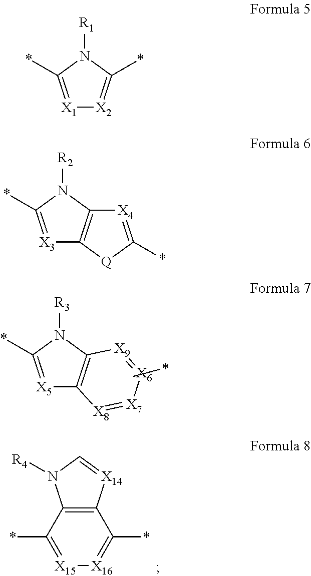

wherein preferably X₁ and X₂ and/or X₃ and X₄ are CR₆, preferably Re is selected from H and alkyl; and/or wherein preferably X₅ is CR₆ with Re being H or alkyl, and/or wherein preferably X₆ to X₉ are CR₆; and/or wherein preferably R₁ to R₃ are independently H, alkyl or aryl; and/or wherein preferably R₄ is H or alkyl.

In a preferred embodiment of the invention, X₁ to X₁₆ are CR₆.

In a preferred embodiment of the invention, T1 is selected from the group consisting of

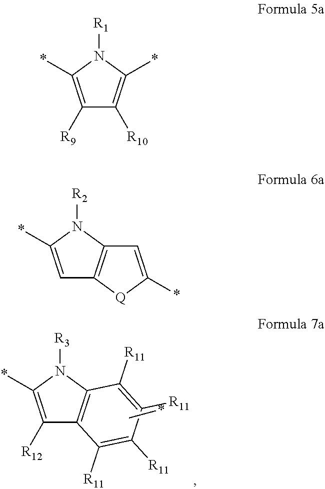

wherein R₉ and R₁₀ are each independently selected from the group consisting of H, halogen, preferably F and Cl, CN, alkyl, alkenyl, O-alkyl, S-alkyl, aryl, heteroaryl; preferably R₉ and R₁₀ are independently H or alkyl, wherein R₁₁ and R₁₂ are each independently selected from the group consisting of H, halogen, preferably F and Cl, CN, alkyl, alkenyl, O-alkyl, S-alkyl, aryl, heteroaryl; preferably R₁₁ and R₁₂ are H.

According to a further development of the invention, it is provided that T2 and T3 are selected from the group consisting of:

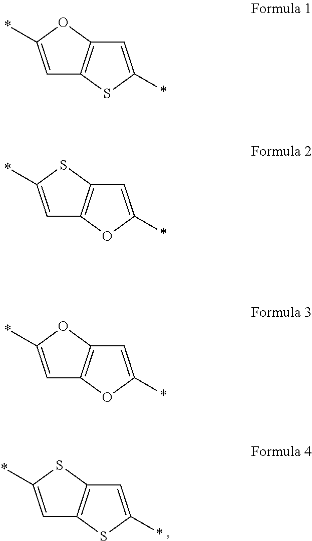 [SMILES: *c1cc2sc(*)cc2o1 ; *c1cc2sc(*)cc2o1 ; *c1cc2oc(*)cc2o1 ; *c1cc2sc(*)cc2s1]

wherein H atoms of Formulas 1 to 4 may be substituted by alkyl, O-alkyl, S-alkyl or halogen; preferably H atoms of Formulas 1 to 4 are not substituted,

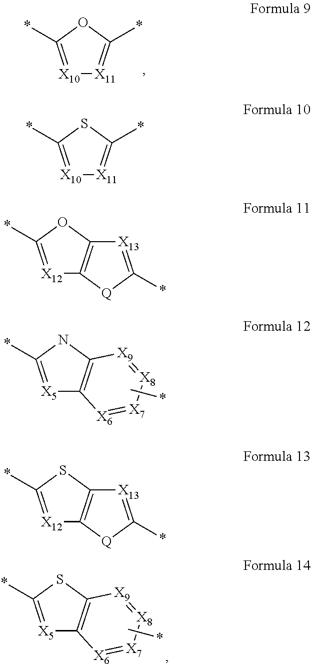

wherein preferably R₆ and R₇ are each independently selected from the group consisting of H, alkyl, and aryl.

In a preferred embodiment of the invention, T3 is selected from the group consisting of

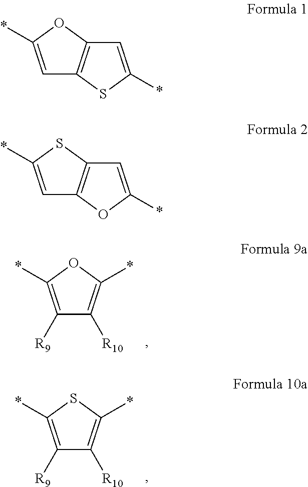

wherein R₉ and R₁₀ are each independently selected from the group consisting of H, halogen, preferably F and Cl, CN, alkyl, alkenyl, O-alkyl, S-alkyl, aryl, heteroaryl; preferably R₉ and R₁₀ are independently H or alkyl.

In a preferred embodiment of the invention, the groups A1, M, T1, T2, T3 and A2 are symmetrical to one another.

According to a development of the invention it is provided that A1 is selected from the group consisting of:

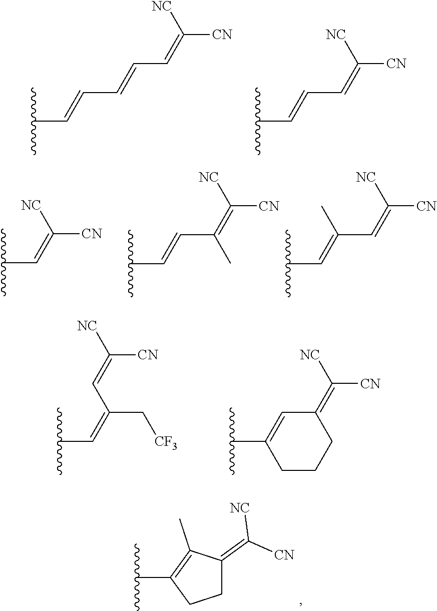

and
A2 is selected from the group consisting of:

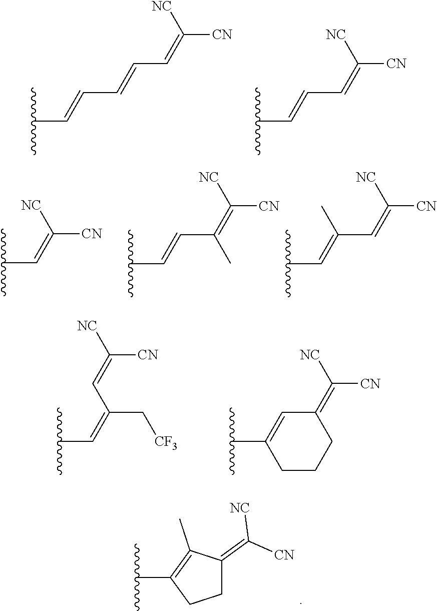

In a preferred embodiment of the invention, the electron-attracting groups A1 and A2 are independently selected from:

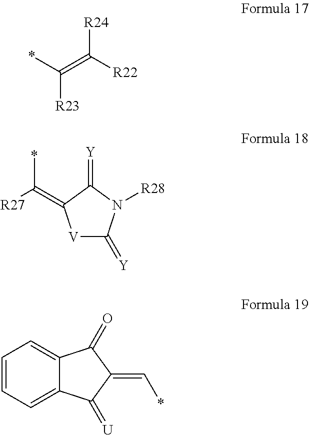

wherein * denotes the link to the groups M, T1, T2 and T3 in the compound of the general formula I,
with R₂₂ and R₂₄ each independently selected from H, CN, and COOR, with the proviso that R₂₂ and R₂₄ are not both H,
wherein R₂₃ is independently selected from the group consisting of H, CN, F, aryl, heteroaryl, alkenyl, alkynyl, alkyl,
with V═O or S; with Y═O, S or C(CN)₂; with U═O, S or C(CN)₂,
wherein R₂₇ and R₂₈ are each independently selected from the group consisting of H, CN, F, aryl, heteroaryl, alkenyl, alkynyl, alkyl, wherein for each of the groups A1 and A2 both the E-isomer and the Z-isomer may be present independently for each C═C double bond.

For each C═C double bond of the formulas 17, 18, and 19, therefore, both the E-isomer (“E”=opposite, i.e., trans-configuration) and also the Z-isomer (“Z”=together, i.e., cis-configuration) are present, wherein these isomers are formed by an imaginary rotation of 180° about the C═C double-bond axis.

In a preferred embodiment of the invention, A1 is equal to A2.

According to a further development of the invention, it is provided that the parameters a, b, c are preferably each 0 or 1 independently of one another, with the proviso that at least one of the parameters a, b, c is equal to 1, preferably at least two of the parameters a, b, c are equal to 1; or the parameter a is equal to 1 or 2, with at least one of the parameters b and c being equal to 1 or 2, preferably with one of the parameters b or c being 1 or 2, preferably with one of the parameters b or c being equal to 1, in particular preferably with the parameter c being equal to 1 or 2.

In a preferred embodiment of the invention, the parameters a, b, c are each independently 0, 1 or 2, with the proviso that at least two of the parameters a, b, c=1.

In a preferred embodiment of the invention, the parameters a, b, c are each independently 0 or 1, with the proviso that at least two of the parameters a, b, c=1.

In a particularly preferred embodiment of the invention, a, b and c are equal to 1.

In a preferred embodiment of the invention, Formulas 1 to 4 of group M are not further fused.

According to a development of the invention it is provided that the group M is selected from the group consisting of:

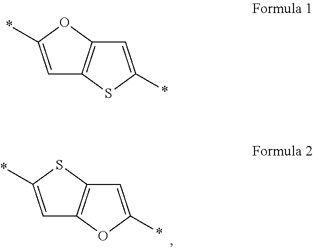 [SMILES: *c1cc2sc(*)cc2o1 ; *c1cc2sc(*)cc2o1]

and
wherein T1 is selected from the group consisting of

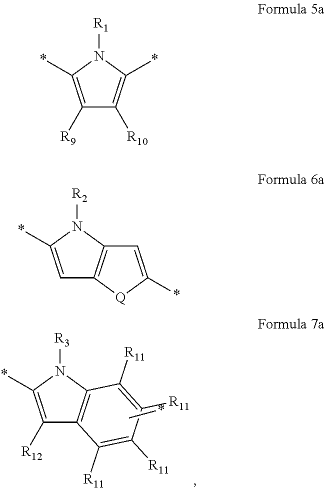

wherein R₉ and R₁₀ are each independently selected from the group consisting of H, halogen, preferably F and Cl, CN, alkyl, alkenyl, O-alkyl, S-alkyl, aryl, heteroaryl; preferably R₉ and R₁₀ are independently H or alkyl,
wherein R₁₁ and R₁₂ are each independently selected from the group consisting of H, halogen, preferably F and Cl, CN, alkyl, alkenyl, O-alkyl, S-alkyl, aryl, heteroaryl; preferably R₁₁ and R₁₂ are H.

According to a further development of the invention, it is provided that the compound has the general formula II

A1-M-(T1)ₐ-(T3)_{c}-A2 (II),
wherein preferably a and c are equal to 1, a is equal to 1 and c is equal to 2, or a is equal to 2 and c is equal to 1.

According to a development of the invention it is provided that the compound be selected from the group consisting of:

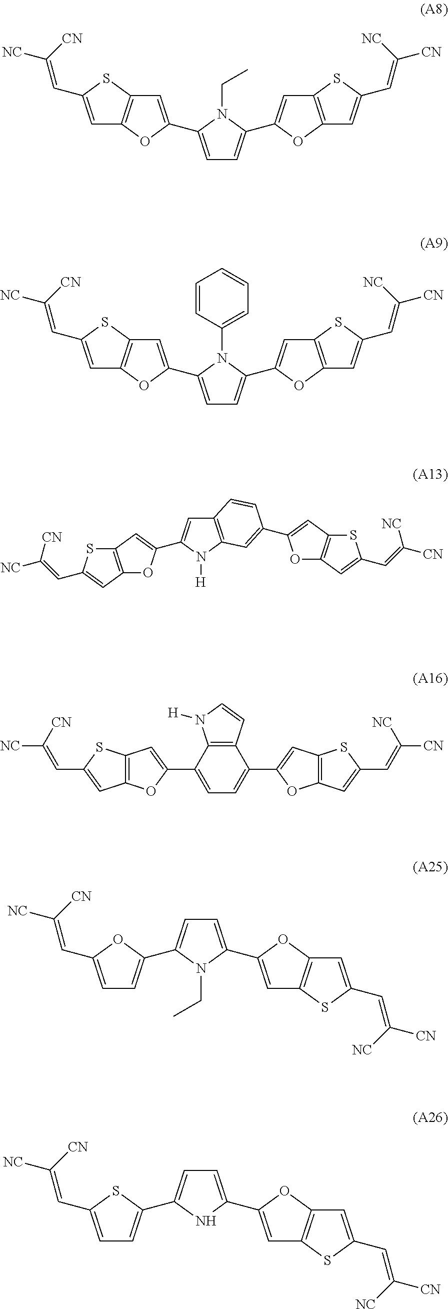 [SMILES: CCn1c(-c2cc3sc(C=C(C#N)C#N)cc3o2)ccc1-c1cc2sc(C=C(C#N)C#N)cc2o1 ; N#CC(C#N)=Cc1cc2oc(-c3ccc(-c4cc5sc(C=C(C#N)C#N)cc5o4)n3-c3ccccc3)cc2s1 ; N#CC(C#N)=Cc1cc2oc(-c3ccc4cc(-c5cc6sc(C=C(C#N)C#N)cc6o5)[nH]c4c3)cc2s1 ; N#CC(C#N)=Cc1cc2oc(-c3ccc(-c4cc5sc(C=C(C#N)C#N)cc5o4)c4[nH]ccc34)cc2s1 ; CCn1c(-c2ccc(C=C(C#N)C#N)o2)ccc1-c1cc2sc(C=C(C#N)C#N)cc2o1 ; N#CC(C#N)=Cc1ccc(-c2ccc(-c3cc4sc(C=C(C#N)C#N)cc4o3)[nH]2)s1]

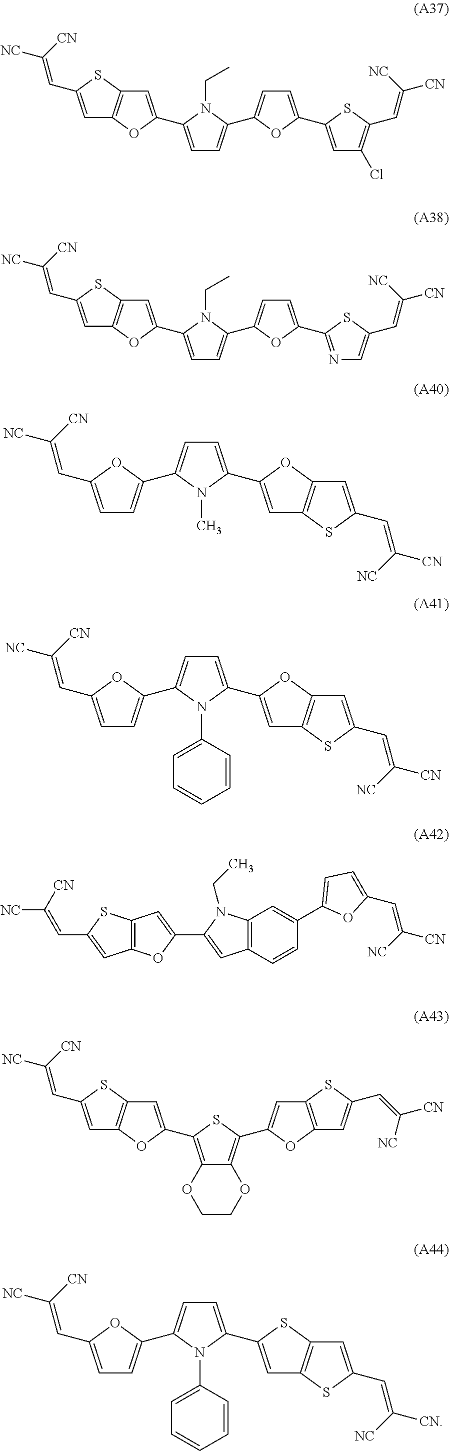

Owing to the particularly strong absorption of the compounds according to the invention, excitons are formed particularly well in layers of electronic devices which comprise these compounds, which, in the case of organic photoactive devices comprising these compounds, leads to higher fill factors FF, improved open-circuit voltage U_{oc}, and improved short-circuit current density J_{sc}. In other organic electronic apparatuses, better electronic values are also to be expected due to the increased charge carrier transport properties of the compounds according to the invention.

The object of the present invention is also achieved by providing a use of at least one compound according to the invention in an organic electronic device, preferably in an organic photovoltaic element, in particular, according to one of the exemplary embodiments described above. In particular, the advantages that have already been explained in conjunction with the compound of the general formula (I) are provided for the use of the compound according to the invention in an organic electronic device.

In a preferred embodiment of the invention, the at least one compound according to the invention is used in an absorber layer of a photovoltaic element.

The object of the present invention is also achieved by providing an organic electronic device comprising an electrode, a counter-electrode and at least one organic photoactive layer between the electrode and the counter-electrode, wherein the at least one organic photoactive layer comprises at least one compound according to the invention, in particular according to one of the previously described exemplary embodiments. In particular, the advantages that have already been explained in conjunction with the compound of the general formula (I) and the use of the compound according to the invention in an organic device are provided for the organic electronic device.

An organic electronic device is understood to mean in particular an electronic device which has organic conductive or semi-conductive materials, and in particular transistors, organic light-emitting devices, organic photoactive apparatuses, in which excitons (electron-hole pairs) can be formed in a photoactive layer by means of irradiation, preferably photodetectors and photovoltaic elements.

According to a development of the invention, it is provided that the organic electronic device is an organic photovoltaic element, an OFET, an OLED, or an organic photodetector. A photovoltaic element is understood to be a solar cell in particular.

In a preferred embodiment of the invention, the organic electronic device is formed as a tandem or multiple cell, wherein at least one further absorber material which absorbs in a different spectral range of light is provided in a further cell. The tandem cell refers here in particular to an electronic device which consists of a vertical layer system of two cells interconnected in series. Accordingly, a multiple cell refers in particular to an electronic device that consists of a vertical layer system of a plurality of cells connected in series.

In a preferred embodiment of the invention, the compound of general formula (I) according to the invention is an absorber material in a photoactive layer of an organic electronic device. In a preferred embodiment of the invention, the compound according to the invention is a donor in a donor-acceptor heterojunction-preferably used with an acceptor selected from the group of fullerenes (C60, C70) or fullerene derivatives, subphthalocyanines, rylenes, fluorenes, carbazoles, benzothiadiazoles, diketopyrrolopyrroles, and vinazenes.

The photoactive layer can be a light-emitting layer which, when a voltage is applied to the electrode and counter-electrode, emits radiation, e.g., light, by recombination of the holes (positive charges) and electrons (negative charge). The photoactive layer can be a light-absorbing layer, wherein excitons (electron-hole pairs) are formed when irradiated with radiation, e.g., visible light, UV radiation or IR radiation. In the case of organic photoactive layers, so-called planar heterojunctions can be formed in particular, in which case a planar p-conducting layer is present adjacent to a planar n-conducting layer, and the excitons formed by irradiation either in the p-conducting or n-conducting layer can be separated into holes and electrons at the interface between the two layers. Furthermore, the photoactive layer can also comprise a so-called bulk heterojunction, in which p-conducting and n-conducting materials transition into one another in the form of an inter-penetrating network, wherein there too the separation of the excitons formed by irradiation occurs at the interfaces between p-conducting and n-conducting materials. Excitons are electrically neutral excitation states, the electron-hole pairs that are then separated in a further step at a p-n junction into electrons and holes. The separation into free charge carriers, which contribute to the electrical current flow, thus takes place. In this case, the size of the band gap of the semi-conductor is limiting, and accordingly only photons which have an energy which is greater than their band gap can be absorbed. As a result of the light, excitons are always produced first, and no free charge carriers; accordingly, the low-recombination diffusion is a component important for the magnitude of the photocurrent. In this case, the exciton diffusion length must exceed the typical penetration depth of the light, so that the largest possible portion of the light can be used electrically. The excitons pass by diffusion to an interface where electrons and holes are separated from one another. The material which absorbs the electrons is referred to as an acceptor, and the material which absorbs the holes is referred to as an electron donor.

A structure of a common organic photovoltaic element which is already known from the literature consists of PIN or NIP diodes [Martin Pfeiffer, “Controlled doping of organic vacuum deposited dye layers: basics and applications,” PhD thesis, TU-Dresden, 1999, and WO 2011/161108 A1]: a pin solar cell in this case consists of a carrier/substrate with, attached to it, the usually transparent base contact, p-layer(s), i-layer(s), n-layer(s), and a cover contact. A nip cell consists of a carrier/substrate with, attached thereto, the usually transparent base contact, n-layer(s), i-layer(s), p-layer(s), and a cover contact. This means n- or p-doping that leads to an increase in the density of free electrons or holes in the thermal equilibrium state. Such layers are thus to be understood primarily as transport layers. The designation, i-layer, characterizes an undoped or intrinsic layer. One or more i-layers can consist here of one material (planar heterojunctions) and of a mixture of two or more materials (bulk heterojunctions) which have an inter-penetrating network.

WO2011161108A1 discloses a photoactive device with an electrode and a counter-electrode, wherein arranged between the electrodes is at least one organic layer system, also with at least two photoactive layer systems, and arranged between the photoactive layer systems are at least two different transport layer systems of the same charge carrier type. The manufacture of such organic electronic devices, in particular organic photovoltaic elements, is well known to those skilled in the art, especially from the cited literature.

The invention is explained in greater detail below with reference to the drawings In the drawings:

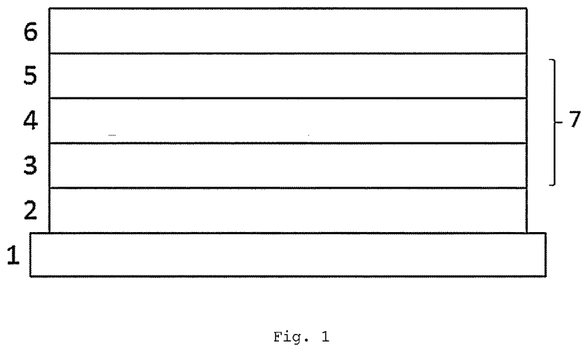

FIG. 1 shows a schematic illustration of an exemplary embodiment of an organic electronic device in cross section;

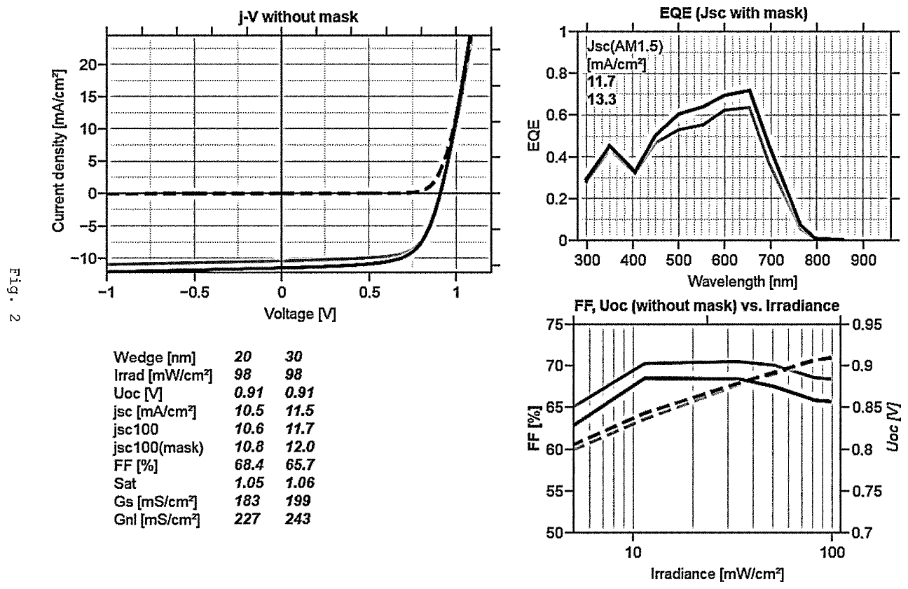

FIG. 2 shows a current-voltage curve of an organic electronic device having the compound A8;

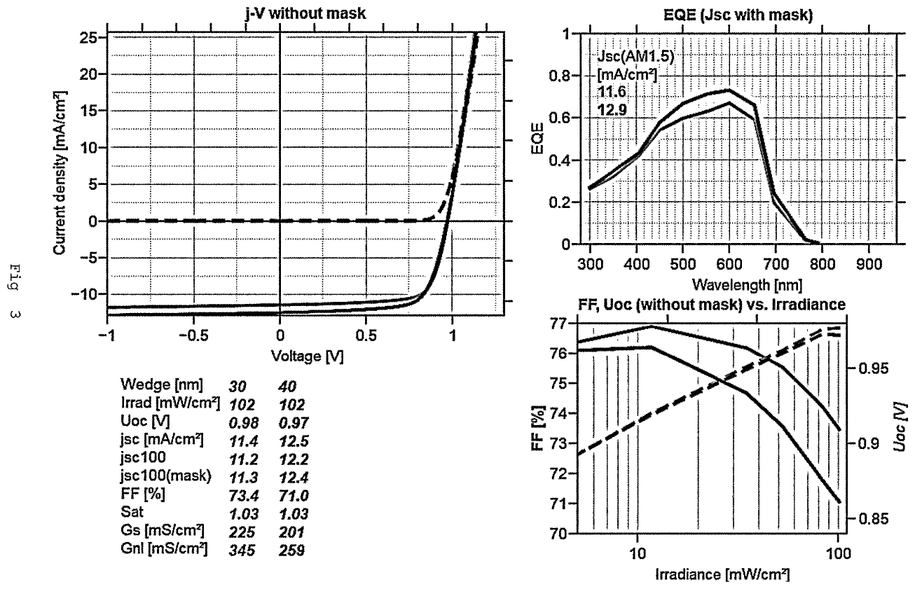

FIG. 3 shows a current-voltage curve of an organic electronic device having the compound A25; and

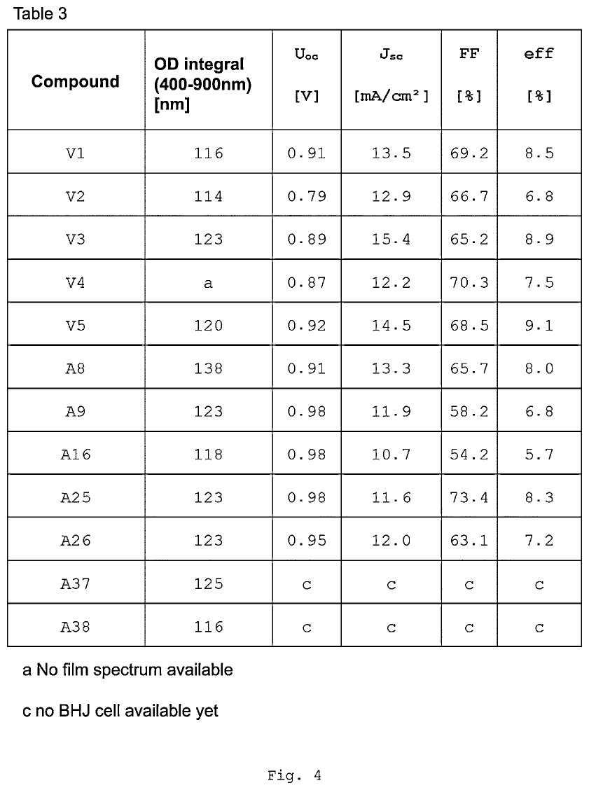

FIG. 4 shows an overview of the parameters fill factor FF, open-circuit voltage Uoc and short-circuit current Jsc of organic electronic devices with compounds according to the invention and compounds not according to the invention in direct comparison.

### EXEMPLARY EMBODIMENTS

FIG. 1 shows a schematic illustration of an exemplary embodiment of an organic electronic device in cross section.

The organic electronic device according to the invention has a layer system 7, wherein at least one layer of the layer system 7 has a compound of the general formula I according to the invention.

The organic electronic component comprises a first electrode 2, a second electrode 6, and a layer system 7, wherein the layer system 7 is arranged between the first electrode 2 and the second electrode 6. Here, at least one layer of the layer system 7 has at least one compound of the general formula I according to the invention.

The layer system 7 has a photoactive layer 4, and preferably a light-absorbing photoactive layer 4, wherein the photoactive layer 4 has the at least one compound according to the invention.

In one exemplary embodiment, the organic photovoltaic element has a substrate 1, e.g., made of glass, on which an electrode 2 is located, which comprises, for example, ITO. Thereupon is arranged the layer system 7 having an electron-transporting layer 3 (ETL) and a photoactive layer 4 having at least one compound according to the invention, a p-conducting donor material, and an n-conducting acceptor material, e.g., C60 fullerene, either as a planar heterojunction or as a bulk heterojunction. Thereover are arranged a p-doped hole transport layer 5 (HTL) and an electrode 6 made of gold or aluminum.

The layer system, and in particular individual layers, of the device can be produced by evaporation of the compounds in a vacuum, with or without carrier gas, or processing of a solution or suspension—for example, during coating or printing. Individual layers can also be applied by sputtering. The production of the layers by evaporation in a vacuum is advantageous, wherein the carrier substrate may be heated. The organic materials are in this case printed onto the films, bonded, coated, applied by vapor deposition, or otherwise applied in the form of thin films or small volumes. All methods which are also used for electronics on glass, ceramic, or semi-conductive carriers are also suitable for the production of the thin layers.

The chemical compound of the general formula I has the following structure:

A1-M-(T1)ₐ-(T2)_{b}-(T3)_{c}-A2 (I)
with the parameters a, b, c in each case independently of one another 0, 1 or 2, with the proviso that at least one of the parameters a, b, c=1 or 2, with group M selected from the group consisting of:

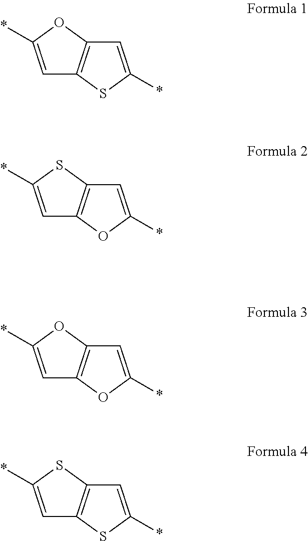 [SMILES: *c1cc2sc(*)cc2o1 ; *c1cc2sc(*)cc2o1 ; *c1cc2oc(*)cc2o1 ; *c1cc2sc(*)cc2s1]

wherein * denotes the link to the groups T1, T2, T3, A1 and A2, wherein H atoms of Formulas 1 to 4 may be substituted by alkyl, O-alkyl, S-alkyl or halogen; with the groups T1, T2 and T3 independently selected from the group consisting of:

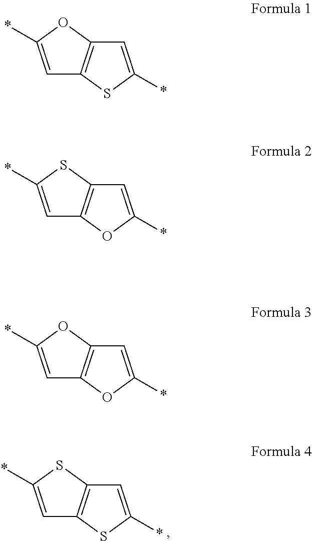 [SMILES: *c1cc2sc(*)cc2o1 ; *c1cc2sc(*)cc2o1 ; *c1cc2oc(*)cc2o1 ; *c1cc2sc(*)cc2s1]

wherein H atoms of Formulas 1 to 4 may be substituted by alkyl, O-alkyl, S-alkyl or halogen,

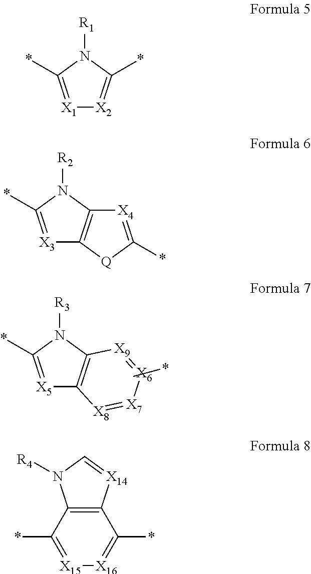

with R₁ to R₄ each independently selected from the group consisting of H, alkyl, alkenyl, aryl and heteroaryl;

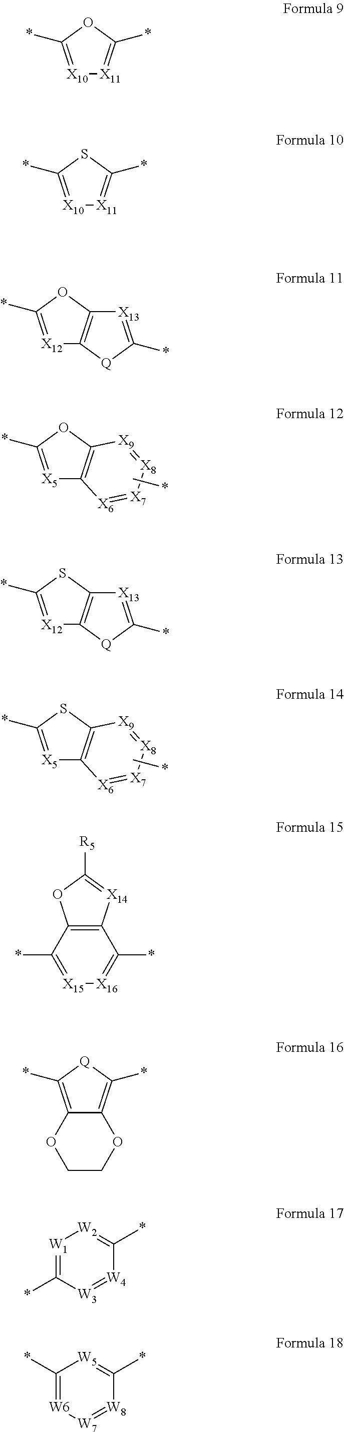

with X₁ to X₁₆, selected independently from N or CR₆, with the proviso that, in the Formulas 7, 12 and 14, a group of X₆ to X₉ is denoted by the link * to a further group in the compound of the general Formula I,
with Q selected from the group consisting of O, S, and NR₇,
with W₁ to W₈ each independently selected from N and CR₈,
wherein R₆, R₇ and R₈ are each independently selected from the group consisting of H, CN, alkyl, alkenyl, O-alkyl, S-alkyl, aryl, and heteroaryl;
wherein the electron-attracting groups A1 and A2 are, independently of one another, electron-attracting groups having at least one C═C double bond, and wherein * denotes the link to a further group M, T1, T2, T3 and A2 in the compound of the general formula I.

In one embodiment of the invention, T1 is independently selected from the group consisting of:

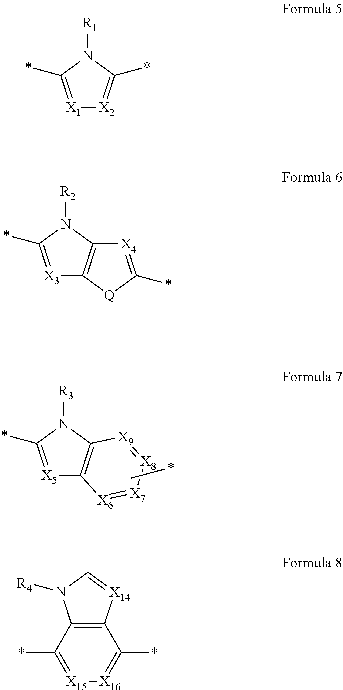

wherein preferably X₁ and X₂ and/or X₃ and X₄ are CR₆, preferably R₆ is selected from H and alkyl; and/or wherein preferably X₅ is CR₆ with R₆ being H or alkyl, and/or wherein preferably X₆ to X₉ are CR₆; and/or wherein preferably R₁ to R₃ are independently H, alkyl or aryl; and/or wherein preferably R₄ is H or alkyl.

In a further embodiment of the invention, T2 and T3 are selected from the group consisting of:

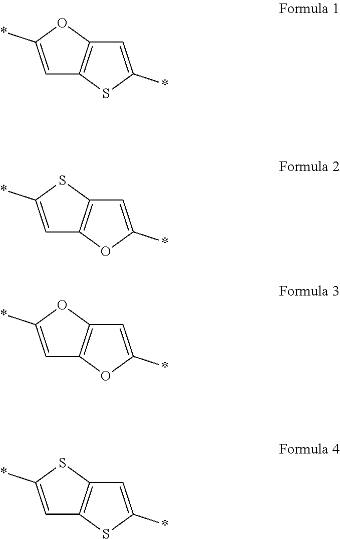 [SMILES: *c1cc2sc(*)cc2o1 ; *c1cc2sc(*)cc2o1 ; *c1cc2oc(*)cc2o1 ; *c1cc2sc(*)cc2s1]

wherein H atoms of Formulas 1 to 4 may be substituted by alkyl, O-alkyl, S-alkyl or halogen; preferably H atoms of Formulas 1 to 4 are not substituted,

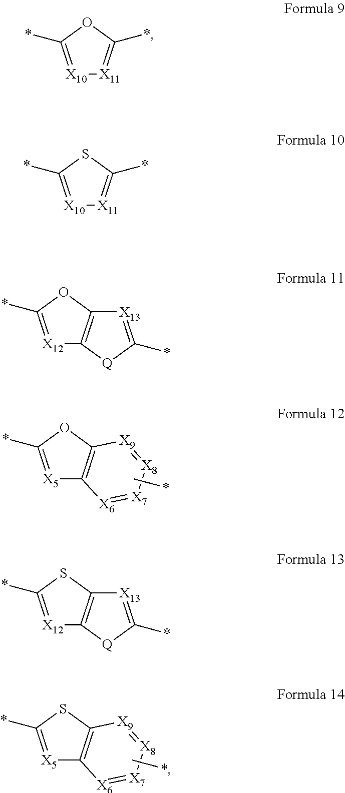

wherein preferably R₆ and R₇ are each independently selected from the group consisting of H, alkyl, and aryl.

In a further embodiment of the invention, A1 is selected from the group consisting of:

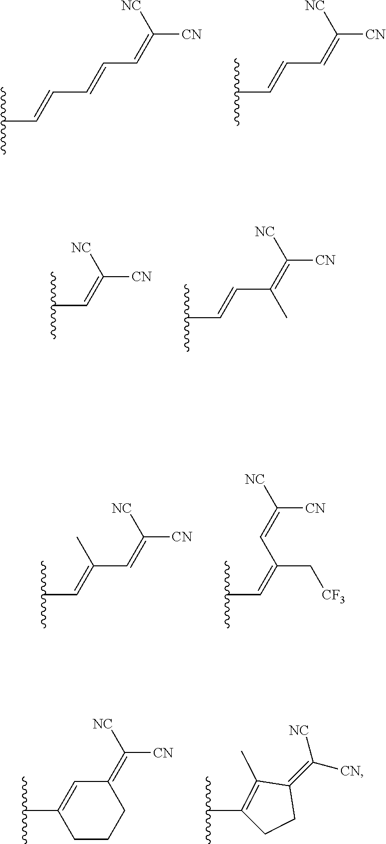

and
A2 is selected from the group consisting of:

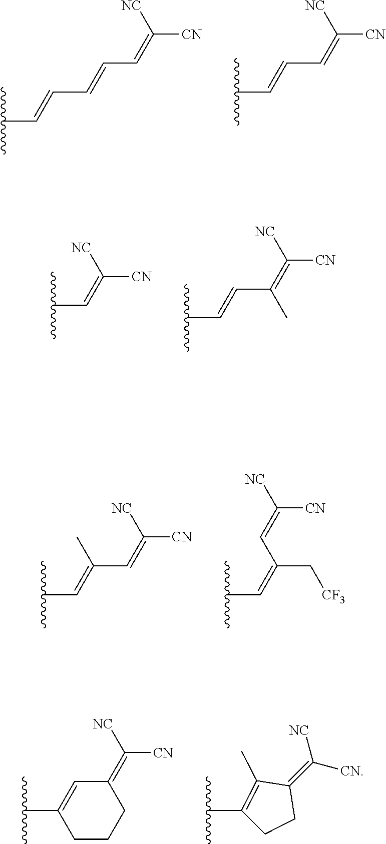

In a further embodiment of the invention, the parameters a, b, c are preferably each independently 0 or 1, with the proviso that at least one of the parameters a, b, c is equal to 1, preferably at least two of the parameters a, b, c are equal to 1; or the parameter a is equal to 1 or 2, with at least one of the parameters b and c being equal to 1 or 2, preferably with one of the parameters b or c being equal to 1 or 2, preferably with one of the parameters b or c being equal to 1, in particular preferably with the parameter c being equal to 1 or 2.

In a further embodiment of the invention, the group M is selected from the group consisting of:

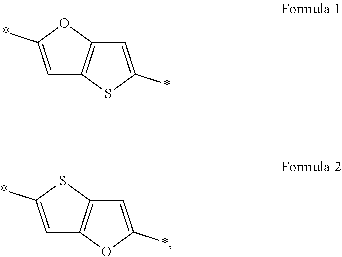

and
wherein T1 is selected from the group consisting of

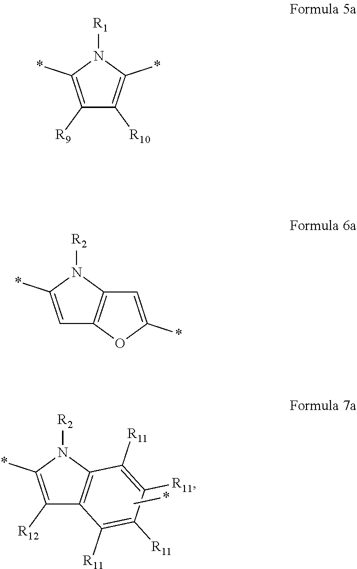

wherein R₉ and R₁₀ are each independently selected from the group consisting of H, halogen, preferably F and Cl, CN, alkyl, alkenyl, O-alkyl, S-alkyl, aryl, heteroaryl; preferably R₉ and R₁₀ are independently H or alkyl,
wherein R₁₁ and R₁₂ are each independently selected from the group consisting of H, halogen, preferably F and Cl, CN, alkyl, alkenyl, O-alkyl, S-alkyl, aryl, heteroaryl; preferably R₁₁ and R₁₂ are H.

In a further embodiment of the invention, the compound has the general formula II

A1-M-(T1)ₐ-(T3)_{c}-A2 (II),
wherein preferably a and c are equal to 1, a is equal to 1 and c is equal to 2, or a is equal to 2 and c is equal to 1.

Table 1 provides an overview of the thermal properties of compounds according to the invention (melting points and thermal budget). The thermal properties show that compounds according to the invention are suitable for manufacturing electronic devices by means of processing in a vacuum.

TABLE 1

| Compound | M.p./° C.^{a} | Thermal budget/° C. ^{b} |
|---|---|---|
| A8 | 339 | 74 |
| A9 | 373 | 118 |
| A16 | 432 | 122 |
| A25 | 282 | 62 |
| A26 | 367 | 107 |
| A37 | 296 | 46 |
| A38 | 267 | 27 |

^{a}onset DSC
^{b} Difference between melting point and evaporation temperature

Table 2 shows an overview of absorption maxima (in nm and eV in the solvent (LM)) of compounds according to the invention.

TABLE 2

| Com- | | M.p./ | λmax | |
|---|---|---|---|---|
| pound | Structure | ° C.ª | (LM)/nm^{b} | in eV |
| A8 | 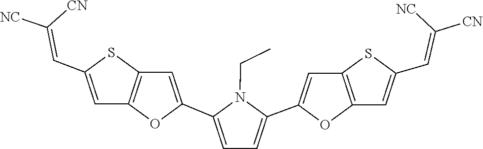 [SMILES: CCn1c(-c2cc3sc(C=C(C#N)C#N)cc3o2)ccc1-c1cc2sc(C=C(C#N)C#N)cc2o1] | 339 | 529 | 2.34 |
| A9 | 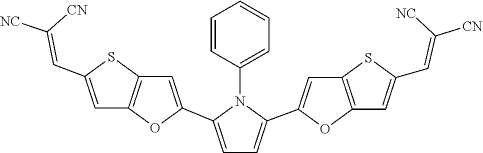 [SMILES: N#CC(C#N)=Cc1cc2oc(-c3ccc(-c4cc5sc(C=C(C#N)C#N)cc5o4)n3-c3ccccc3)cc2s1] | 373 | 574 | 2.16 |
| A13 | 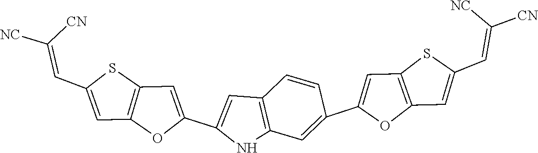 [SMILES: N#CC(C#N)=Cc1cc2oc(-c3ccc4cc(-c5cc6sc(C=C(C#N)C#N)cc6o5)[nH]c4c3)cc2s1] | 362 | 540 | 2.30 |
| A16 | 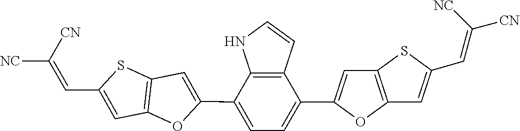 [SMILES: N#CC(C#N)=Cc1cc2oc(-c3ccc(-c4cc5sc(C=C(C#N)C#N)cc5o4)c4[nH]ccc34)cc2s1] | 432 | 535 | 2.32 |
| A25 | 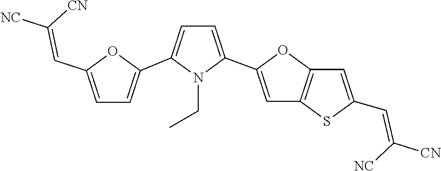 [SMILES: CCn1c(-c2ccc(C=C(C#N)C#N)o2)ccc1-c1cc2sc(C=C(C#N)C#N)cc2o1] | 282 | 543 | 2.28 |
| A26 | 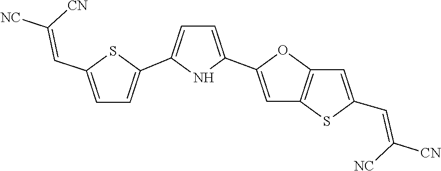 [SMILES: N#CC(C#N)=Cc1ccc(-c2ccc(-c3cc4sc(C=C(C#N)C#N)cc4o3)[nH]2)s1] | 367 | 543 | 2.28 |
| A37 | 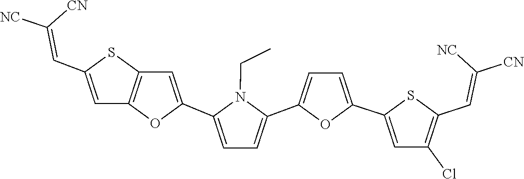 [SMILES: CCn1c(-c2ccc(-c3cc(Cl)c(C=C(C#N)C#N)s3)o2)ccc1-c1cc2sc(C=C(C#N)C#N)cc2o1] | 296 | 571 | 2.17 |
| A38 | 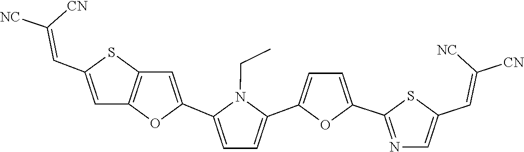 [SMILES: CCn1c(-c2ccc(-c3ncc(C=C(C#N)C#N)s3)o2)ccc1-c1cc2sc(C=C(C#N)C#N)cc2o1] | 267 | 543 | 2.28 |

^{a}onset DSC (differential scanning calorimetry; start of the melting range; extrapolated start temperature (intersection point inflectional tangent baseline)
^{b}in dichloromethane unless otherwise noted

Surprisingly, it has been found that the compounds according to the invention have particularly strong absorption, i.e., a high optical density at the absorption maximum and/or a high integral over the optical density in the visible spectral range compared to similar compounds outside the range claimed here.

It was also shown that the compounds according to the invention not only absorb light, but can also be evaporated without residue in a vacuum. High photocurrents can be produced by very good charge transport properties and good absorption properties. Thus, very well combined tandem/triple/quadruple/or multi-junction cells can be produced.

FIG. 2 shows a current-voltage curve of an organic electronic device having the compound A8. In this exemplary embodiment, the organic electronic device is an organic photovoltaic element.

The photovoltaic parameters U_{oc}, Jsc and FF each refer to photovoltaic elements with a 30 nm thick mixed layer made of the respective donor material of these compounds and fullerene C60 as a photoactive layer formed as a bulk heterojunction (BHJ) on glass with the structure:

ITO/C60 (15 nm)/compound A8:C60 (30 nm, 3:2, 40° C.)/NHT169 (10 nm)/NHT169:NDP9 (30 nm, 9.1 wt %)/NDP9 (1 nm)/Au (50 nm), wherein the photoactive layer is a bulk heterojunction (BHJ). Here, ITO is indium tin oxide, NDP9 is a commercial p-dopant of Novaled GmbH, and NHT169 is a commercial hole conductor of Novaled GmbH. The parameters of the cell were measured under AM1.5 illumination (Am=Air Mass; in this spectrum the global radiant power is 1000 W/m²; AM=1.5 as the standard value for measuring solar modules).

In the organic photovoltaic element having the compound A8, the fill factor FF is 65.7%, the open-circuit voltage Uoc is 0.91 V, and the short-circuit current Jsc is 13.3 mA/cm2. The cell efficiency of the photovoltaic element with compound A8 is 8.0%.

FIG. 3 shows a current-voltage curve of an organic electronic device having the compound A25. In this exemplary embodiment, the organic electronic device is an organic photovoltaic element.

The structure of the photovoltaic element corresponds to that shown in FIG. 2:
ITO/C60 (15 nm)/compound A25:C60 (30 nm, 3:2, 40° C.)/NHT049 (10 nm)/NHT049:NDP 9 (30 nm, 9.1 wt %)/NDP9 (1 nm)/Au (50 nm), wherein the photoactive layer is a bulk heterojunction (BHJ). Here, ITO is indium tin oxide, NDP9 is a commercial p-dopant of Novaled GmbH, and NHT049 is a commercial hole conductor of Novaled GmbH. The parameters of the cell were measured under AM1.5 illumination (Am=Air Mass; in this spectrum the global radiant power is 1000 W/m²; AM=1.5 as the standard value for measuring solar modules).

In the photovoltaic element having the compound A25, the fill factor FF is 73.4%, the open-circuit voltage Uoc is 0.98 V, and the short-circuit current Jsc is 11.6 mA/cm2. The cell efficiency of the photovoltaic element with compound A25 is 8.3%.

FIG. 4 shows an overview of the parameters fill factor FF, open-circuit voltage Voc, and short-circuit current Jsc of organic electronic devices with compounds according to the invention and compounds not according to the invention in direct comparison. In this exemplary embodiment, the organic electronic devices are organic photovoltaic elements.

The structure of the photovoltaic elements corresponds to the structure of the photovoltaic element in FIG. 2 and FIG. 3. FIG. 4 shows parameters of compounds according to the invention and compounds not according to the invention in direct comparison. The photovoltaic parameters refer in each case to photovoltaic elements with a 30 nm thick mixed layer made of the respective donor material of the corresponding compound and fullerene C60 on glass with the structure:
ITO/C60 (15 nm)/compound:C60 (30 nm, 3:2, 40° C.)/NHT049 or NHT169 (10 nm)/NHT049 or NHT169:NDP9 (30 nm, 9.1 wt %)/NDP9 (1 nm)/Au (50 nm), wherein the photoactive layer is a bulk heterojunction (BHJ).

The comparative compounds not according to the invention V1 to V5 are as follows:

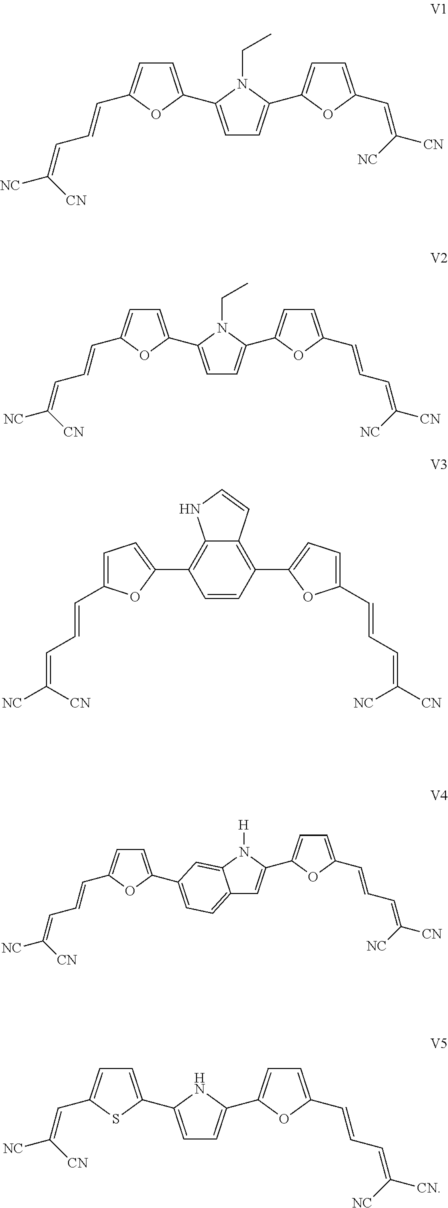 [SMILES: CCn1c(-c2ccc(C=C(C#N)C#N)o2)ccc1-c1ccc(/C=C/C=C(C#N)C#N)o1 ; CCn1c(-c2ccc(/C=C/C=C(C#N)C#N)o2)ccc1-c1ccc(/C=C/C=C(C#N)C#N)o1 ; N#CC(C#N)=C/C=C/c1ccc(-c2ccc(-c3ccc(/C=C/C=C(C#N)C#N)o3)c3[nH]ccc23)o1 ; N#CC(C#N)=C/C=C/c1ccc(-c2ccc3cc(-c4ccc(/C=C/C=C(C#N)C#N)o4)[nH]c3c2)o1 ; N#CC(C#N)=C/C=C/c1ccc(-c2ccc(-c3ccc(C=C(C#N)C#N)s3)[nH]2)o1]

The parameters of organic electronic devices parameters with compounds according to the invention and compounds not according to the invention are summarized in Table 3 (FIG. 4). Table 3 shows the optical integral in the visible range (OD integral), and U_{oc}, Jsc, FF, and the efficiency. Compared to the compounds not according to the invention (V1 to V5), the compounds according to the invention show in particular an increased open-circuit voltage Uoc of 0.91 V and higher, in particular in a spectral range from 400 nm to 600 nm.

In the following, syntheses of specific exemplary embodiments of compounds according to the invention are shown by way of example.

The general compound (I) can be synthesized by one of the methods described below. This is intended here to be an exemplary representation and can be varied in the sequence of its individual steps, or can be modified by other known methods. A combination of individual reaction steps or a change in parts of the synthesis route is also possible.

### Synthesis Group 1

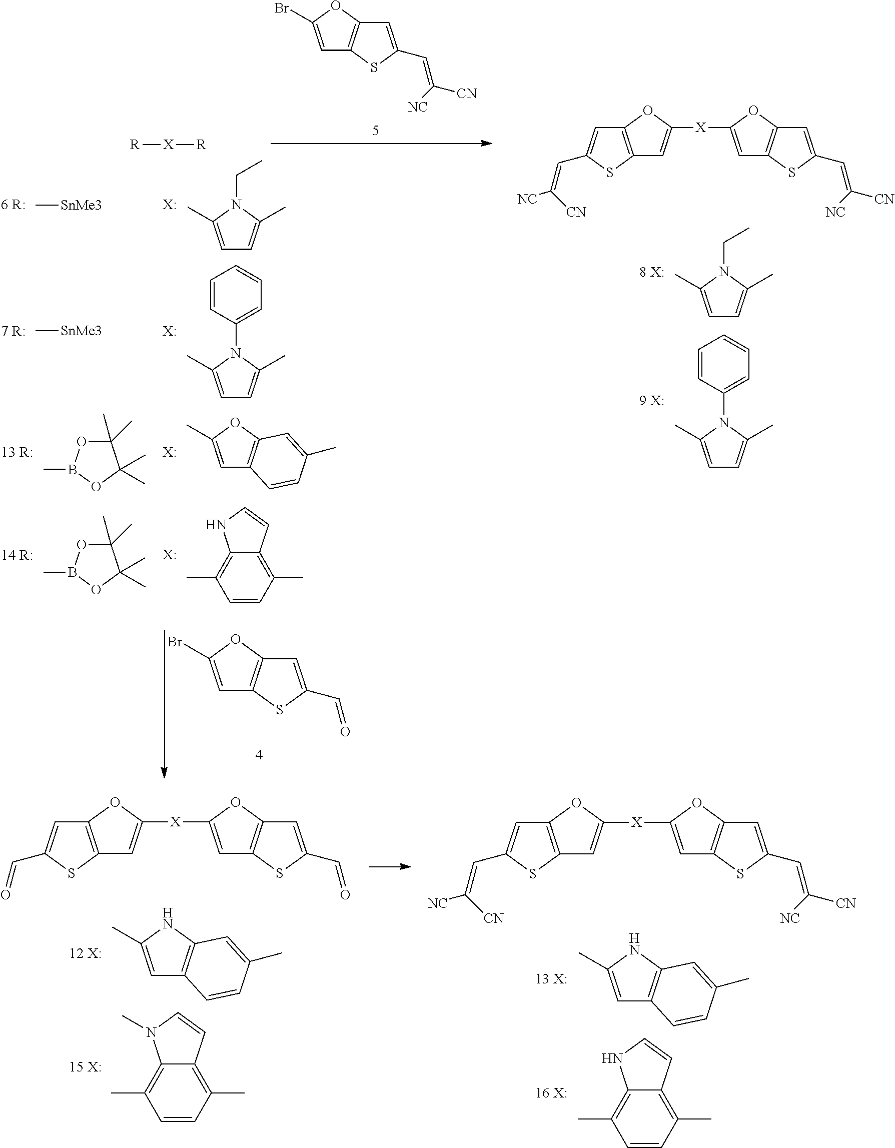

### Synthesis of Compound A8 and Compound A9

### Synthesis of 2,2′ {(1-ethyl-1H-pyrrole-2,5-diyl)bis[(thieno[3,2-b]furan-2,5-diyl)methanylylidene]}dipropanedinitriles (A8) and 2,2′-{(1-phenyl-1H-pyrrole-2,5-diyl)bis[(thieno[3,2-b]furan-2,5-diyl) methanylylidene]}dipropanedinitriles (A9)

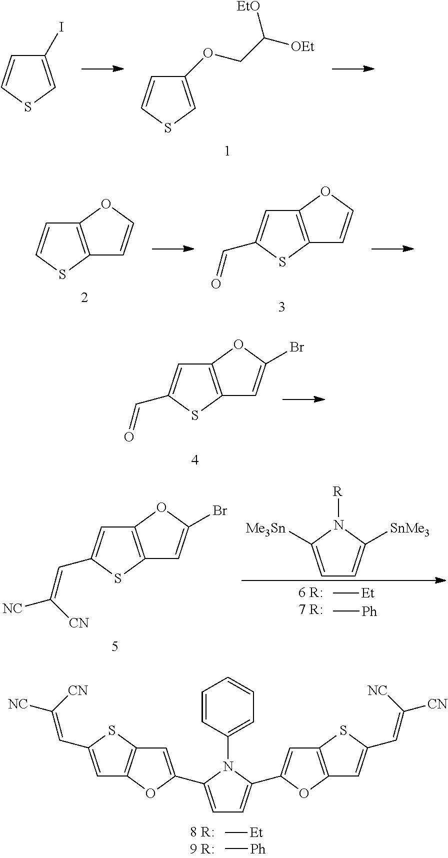

Compounds 6 and 7 could be synthesized as described by Fitzner, Roland; Gerdes, Olga; Hildebrandt, Dirk; D'Souza, Daniel; Mattersteig, Gunter; Weiss, Andre From Eur. Pat. Appl. (2017), EP3188270A1.

### Synthesis of 3-(2,2-diethoxyethoxy)thiophene (1)

3.28 g (15 mmol) of 3-iodothiophene, 4.11 g (30 mmol) of 2,2-diethoxyethanol and 601 mg (3 mmol) of 1,10-phenanthroline monohydrate was dissolved in 7.5 ml of toluene. 286 mg (1.5 mmol) of copper (I) iodide and 9.77 g (30 mmol) of caesium carbonate was added. The reaction mixture was heated to boiling for 64 hours. After the mixture had cooled down, it was mixed with diethyl ether and filtered through silica gel. The filtrate was concentrated and the crude product was chromatographed on silica gel in diethyl ether:petroleum ether 1:9 (Rf 0.43). In the process, 2.31 g (71% yield) was isolated from 3-(2,2-diethoxyethoxy)thiophene 1. GC-MS (EI) m/z: 216 (20%), 171 (20%), 125 (60%), 103 (100%). 1H-NMR in CDCl3, ppm: 7.16 (dd, 1H), 6.78 (dd, 1H), 6.26 (dd, 1H), 4.82 (t, 1H), 3.99 (d, 2H), 3.75 (m, 2H), 3.62 (m, 2H), 1.25 (t, 6H).

### Synthesis of thieno[3,2-b]furan (2)

2.31 g (10.7 mmol) of 3-(2,2-diethoxyethoxy)thiophene 1 was dissolved in 54 ml THF and 2.4 g of Amberlyst 15 was added. The mixture was heated to boiling for 10 hours. After the mixture had cooled down, it was decanted. Amberlyst® 15 remaining in the residue was stirred in hot THF and then decanted again. The combined organic phases were diluted with petroleum ether, washed with sat. NaCl solution and dried over sodium sulfate. After the solvents were removed on the rotary evaporator, the residue was purified by column chromatography on silica gel in petroleum ether. In the process, 448 mg (34% yield) was isolated from thieno[3,2-b]furan 2. GC-MS (EI) m/z: 124 (100%). 1H-NMR in d6-acetone, ppm: 7.74 (dd, 1H), 7.41 (dd, 1H), 7.15 (dd, 1H), 6.90 (dd, 1H).

### Synthesis of thieno[3,2-b]furan-5-carbaldehyde (3)

10.1 g (81.1 mmol) of thieno[3,2-b]furan 2 was dissolved in 143 ml dry THF and the solution was cooled to −78° C. 32.4 mL (81.1 mmol) of 2,5-M-tBuLi was slowly added, and the mixture was stirred at −78° C. for 2 h. 9.27 g (81.1 mmol) of N-formylpiperidine was added to the reaction mixture, and the reaction mixture was allowed to warm to room temperature overnight. The reaction mixture was mixed with sat. NaHCO₃ solution and extracted with dichloromethane. Organic phase was washed with water and sat. NaCl solution and dried over sodium sulfate. Crude product was purified by column chromatography on silica gel in petroleum ether:dichloromethane 1:3. In the process, 7.8 g (63% yield) was isolated from thieno[3,2-b]furan-5-carbaldehyde 3. GC-MS (EI) m/z: 152 (100%), 123 (20%). 1H-NMR in d6-acetone, ppm: 9.95 (s, 1H), 8.06 (s, 1H), 8.04 (dd, 1H), 7.09 (dd, 1H).

### Synthesis of 2-bromothieno[3,2-b]furan-5-carbaldehyde (4)

1.52 g (10 mmol) of 2-bromothieno[3,2-b]furan-5-carbaldehyde 4 was dissolved in 23 ml dry DMF. 2.72 g (15 mmol) of N-bromosuccinimide was added in small portions, and the reaction mixture was stirred at room temperature for 1 h. The mixture was then placed on ice and any precipitate was filtered off. Crude product was recrystallized from ethanol. In the process, 1.74 g (75% yield) was isolated from 2-bromothieno[3,2-b]furan-5-carbaldehyde 4. GC-MS (EI) m/z: 231 (100%). 1H-NMR in d6-acetone, ppm:

9.97 (s, 1H), 8.04 (s, 1H), 7.19 (s, 1H).

### Synthesis of [(2-bromothieno[3,2-b]furan-5-yl)methylidene]propanedinitrile (5)

690 mg (2.98 mmol) of [(2-bromothieno[3,2-b]furan-5-yl)methylidene]propanedinitrile 5 and 394 mg (5.97 mmol) of malodinitrile was dissolved in 8 ml of ethanol and 13.6 mg (0.15 mmol) of β-alanine was added. The reaction mixture was heated to boiling for 2 hours. After the mixture had cooled to room temperature, the precipitate was filtered off and washed with a little ethanol. This yielded 555 mg (67% yield) of [(2-bromothieno[3,2-b]furan-5-yl)methylidene]propanedinitrile 5. GC-MS (EI) m/z: 279 (55%). 1H-NMR in d6-acetone, ppm: 8.46 (s, 1H), 7.99 (s, 1H), 7.29 (1H).

Synthesis of 2,2′-{(1-ethyl-1H-pyrrole-2,5-diyl)bis[(thieno[3,2-b]furan-2,5-diyl)methanylylidene]}dipropanedinitriles (A8) and 2,2′-{(1-phenyl-1H-pyrrole-2,5-diyl)bis[(thieno[3,2-b]furan-2,5-diyl)methanylylidene]}dipropanedinitriles (A9), general protocol.

1 eq of bisstannyl compound 6 or 7 and 2 eq of [(2-bromothieno[3,2-b]furan-5-yl)methylidene]propanedinitrile 5 were dissolved in dioxane, and the solution was degassed. 0.033 eq of bis-(tri-tert-butylphosphine)-palladium (0) was added, and the reaction mixture was heated to 60° C. overnight. After the reaction mixture had cooled to room temperature, the precipitate was filtered and washed with methanol. The crude product was recrystallized from chlorobenzene and then sublimated.
8: 33% yield, HPLC purity 98.5% @528 nm
9: 16% yield, HPLC purity 100% @574 nm

### Synthesis compound A13

### Synthesis of 2,2′ {1H-indole-2,6-diylbis[(thieno[3,2-b]furan-2,5-diyl)methanylylidene]}dipropanedinitriles (A13)

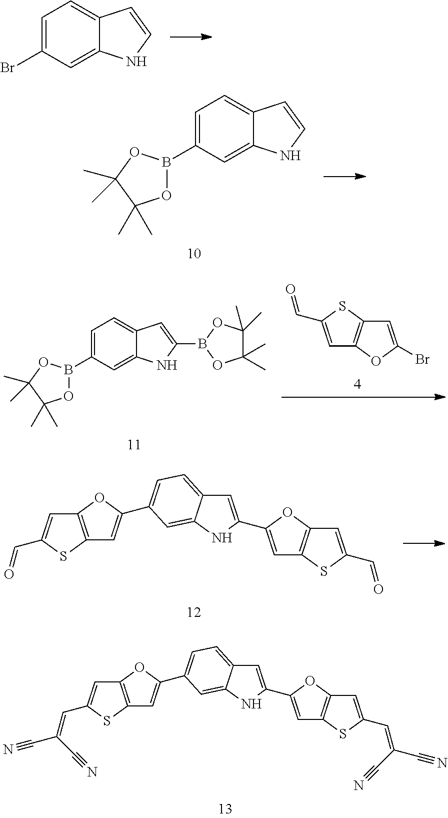

### Synthesis of 6-(4,4,5,5-tetramethyl-1,3,2-dioxaborolan-2-yl)-1H-indole (10)

3.92 g (20 mmol) of 6-bromoindole was dissolved in 140 ml dioxane. 5.59 g (22 mmol) of bis-(pinacolato)-diboron and 5.89 g (60 mmol) of potassium acetate was added, and the reaction mixture was degassed. 293 mg (0.4 mmol) of 1,1′-bis(diphenylphosphino) ferrocene-palladium(II) dichloride was added to the reaction mixture, and it was stirred overnight at 90° C. After the reaction mixture had cooled to room temperature, 2 molar hydrochloric acid solution was added. It was extracted with dichloromethane; the organic phase was extracted with sat. ammonium chloride solution, water and sat. NaCl solution. The crude product was purified by silica gel filtration in dichloromethane, wherein 1.49 g (31% yield) was cleanly isolated from 6-(4,4,5,5-tetramethyl-1,3,2-dioxaborolan-2-yl)-1H-indole 10. GC-MS (EI), m/z: 243 (100%), 228 (40%). 1H-NMR in d6-acetone, ppm: 10.31 (sb, 1H), 7.89 (m, 1H), 7.57 (m, 1H), 7.43-7.40 (m, 2H), 6.48 (s, 1H), 1.21 (s, 12H).

### Synthesis of 2,6-bis(4,4,5,5-tetramethyl-1,3,2-dioxaborolan-2-yl)-1H-indoles (11)

0.78 g (3.07 mmol) of bis-(pinacolato)-diboron, 60 mg (0.22 mmol) of 4,4-di-tert-butyl-2,2-dipyridyl and 61 mg (0.09 mmol) of 1,5-cyclooctadiene)(methoxy) iridium(I) dimer was dissolved in 31 ml of tetrahydrofuran. 1.49 g (6.14 mmol) of 6-(4,4,5,5-tetramethyl-1,3,2-dioxaborolan-2-yl)-1H-indole 10 was added to the reaction mixture and it was stirred at 80° C. overnight. After the reaction mixture had cooled to room temperature, dichloromethane was added to the reaction mixture, and the organic phase was mixed with water and sat. NaCl solution. The crude product was purified by column chromatography on silica gel with 5% acetone in dichloromethane followed by recrystallization from ethanol, wherein 420 mg (18% yield) was isolated from 2,6-bis(4,4,5,5-tetramethyl-1,3,2-dioxaborolan-2-yl)-1H-indole 11. 1H-NMR in d6-acetone, ppm: 10.45 (s, 1H), 8.00 (s, 1H), 7.60 (d, 1H), 7.41 (d, 1H), 6.99 (s, 1H).

### Synthesis of 2,2′-(1H-indole-2,6-diyl)di(thieno[3,2-b]furan-5-carbaldehydes) (12)

443 mg (1.2 mmol) of 2,6-bis(4,4,5,5-tetramethyl-1,3,2-dioxaborolan-2-yl)-1H-indole 11 was dissolved in 9 ml ethanol. 566 mg (2.4 mmol) of 2-bromothieno[3,2-b]furan-5-carbaldehyde 4, 403 mg (4.8 mmol) of sodium hydrogen carbonate and water was added to the reaction mixture and it was degassed. 65 mg (0.12 mmol) of bis-(tri-tert-butylphosphine)-palladium (0) was added, and the reaction mixture was stirred overnight at 80° C. After the reaction mixture had cooled to room temperature, the precipitate was filtered and washed with methanol. The crude product was recrystallized from toluene, wherein 280 mg (56% yield) was isolated from 2,2′-(1H-indole-2,6-diyl)di(thieno[3,2-b]furan-5-carbaldehyde) 12. HPLC purity: 85.8% @ 436 nm. 1H-NMR in d6-DMSO at 80° C., ppm: 11.97 (sb, 1H), 9.92 (s, 1H), 9.91 (s, 1H), 8.18 (s, 1H), 8.16 (s, 1H), 7.91 (m, 1H), 7.74 (m, 1H), 7.61 (m, 1H), 7.53 (m, 2H), 7.08 (s, 1H).

### Synthesis of 2,2′-{1H-indole-2,6-diylbis[(thieno[3,2-b]furan-2,5-diyl)methanylylidene]}dipropanedinitriles (A13)

280 mg (0.67 mmol) of 2,2′-(1H-indole-2,6-diyl)di(thieno[3,2-b]furan-5-carbaldehyde) 12 was dissolved in 7 ml of dimethyl sulfoxide. 134 mg (2.01 mmol) of malodinitrile and 7.5 mg (0.07 mmol) of 1,4-diazabicyclo[2,2,2]octane was added, and the reaction mixture was stirred overnight at room temperature. The precipitate was filtered and washed with methanol. The crude product was recrystallized from chlorobenzene, 170 mg (49% yield) was isolated from 2,2′-{1H-indole-2,6-diylbis[(thieno[3,2-b]furan-2,5iyl)methanylylidene]}dipropanedinitrile 13. Further purification was carried out by sublimation.

### Synthesis Compound A16

### Synthesis of 2,2′-{1H-indole-4,7-diylbis[(thieno[3,2-b]furan-2,5-diyl)methanylylidene]}dipropanedinitriles (A16)

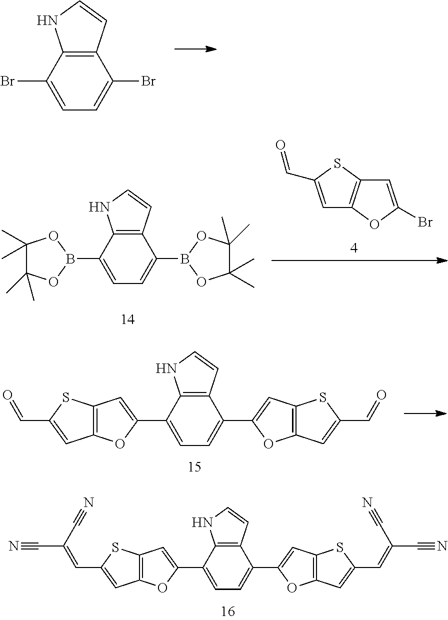

### Synthesis of 4,7-bis(4,4,5,5-tetramethyl-1,3,2-dioxaborolan-2-yl)-1H-indoles (14)

1.37 g (5 mmol) of 4,7-dibromo-1H-indole was dissolved in 35 ml dioxane. 2.79 g (11 mmol) of bis-(pinacolato)-diboron and 2.94 g (30 mmol) of potassium acetate was added and degassed. 146 mg (0.2 mmol) of 1,1′-bis(diphenylphosphino) ferrocene-palladium(II) dichloride was added to the reaction mixture and it was stirred overnight at 90° C. After the reaction mixture had cooled to room temperature, 2 molar hydrochloric acid solution was added. It was extracted with dichloromethane; the organic phase was extracted with sat. ammonium chloride solution, water and sat. NaCl solution. The crude product was purified by silica gel filtration in 50% petroleum ether in dichloromethane, wherein 1.34 g (73% yield) was cleanly isolated from 4,7-bis(4,4,5,5-tetramethyl-1,3,2-dioxaborolan-2-yl)-1H-indole 14. 1H-NMR in d6-acetone, ppm: 9.94 (s, 1H), 7.54 (s, 2H), 7.36 (dd, 1H), 6.92 (dd, 1H), 1.38 (t, 24H).

### Synthesis of 2,2′-(1H-indole-4,7-diyl)di(thieno[3,2-b]furan-5-carbaldehydes) (15)

1.17 g (3.17 mmol) of 4,7-bis(4,4,5,5-tetramethyl-1,3,2-dioxaborolan-2-yl)-1H-indole 14 was dissolved in 9 ml of dioxene. 1.46 g (6.37 mmol) 4, 5.38 g (25.4 mmol) potassium phosphate and 3 ml water was added, and the reaction mixture was degassed. 366 mg (0.32 mmol) of tetrakis-(triphenylphosphine) palladium (0) was added, and the mixture was stirred overnight at 80° C. After the reaction mixture had cooled to room temperature, the precipitate was filtered and washed with water and methanol. The crude product was recrystallized from toluene, wherein 434 mg (33% yield) was isolated from 2,2′-(1H-indole-4,7-diyl)di(thieno[3,2-b]furan-5-carbaldehyde) 15. 1H-NMR in d6-dimethylsulfoxide at 100° C., ppm: 11.71 (sb, 1H), 9.98 (s, 1H), 9.93 (s, 1H), 8.29 (s, 1H), 8.22 (s, 1H), 7.83-7.80 (m, 3H), 7.78 (d, 1H), 7.72 (dd, 1H), 7.17 (dd, 1H).

### Synthesis of 2,2′-{1H-indole-4,7-diylbis[(thieno[3,2-b]furan-2,5-diyl)methanylylidene]}dipropanedinitriles (A16)

434 mg (1.04 mmol) of 2,2′-(1H-indole-4,7-diyl)di(thieno[3,2-b]furan-5-carbaldehyde) 15 was dissolved in 7 ml of dimethyl sulfoxide. 694 mg (10.4 mmol) of malodinitrile and 12 mg (0.01 mmol) of 1,4-diazabicyclo[2,2,2]octane was added, and the reaction mixture was stirred for 5 h. The precipitate was filtered and washed with methanol. The crude product was recrystallized from chlorobenzene and sublimed, 132 mg (25% yield, HPLC purity 99.5% @ 541 nm) was isolated from 2,2′ {1H-indole-4,7-diylbis[(thieno[3,2-b]furan-2,5-diyl)methanylylidene]}dipropanedinitrile A16.

### Synthesis Group 2

### Synthesis of compound A25 and compound A26

### Synthesis of [(2-{5-[5-(2,2-dicyanoethenyl) furan-2-yl]-1-ethyl-1H-pyrrol-2-yl}thieno[3,2-b]furan-5-yl)methylidene]propanedinitriles (A25) and [(5{5-[5-(2,2-dicyanoethenyl) thieno[3,2-b]furan-2-yl]-1H-pyrrol-2-yl}thiophen-2-yl)methylidene]propanedinitriles (A26)

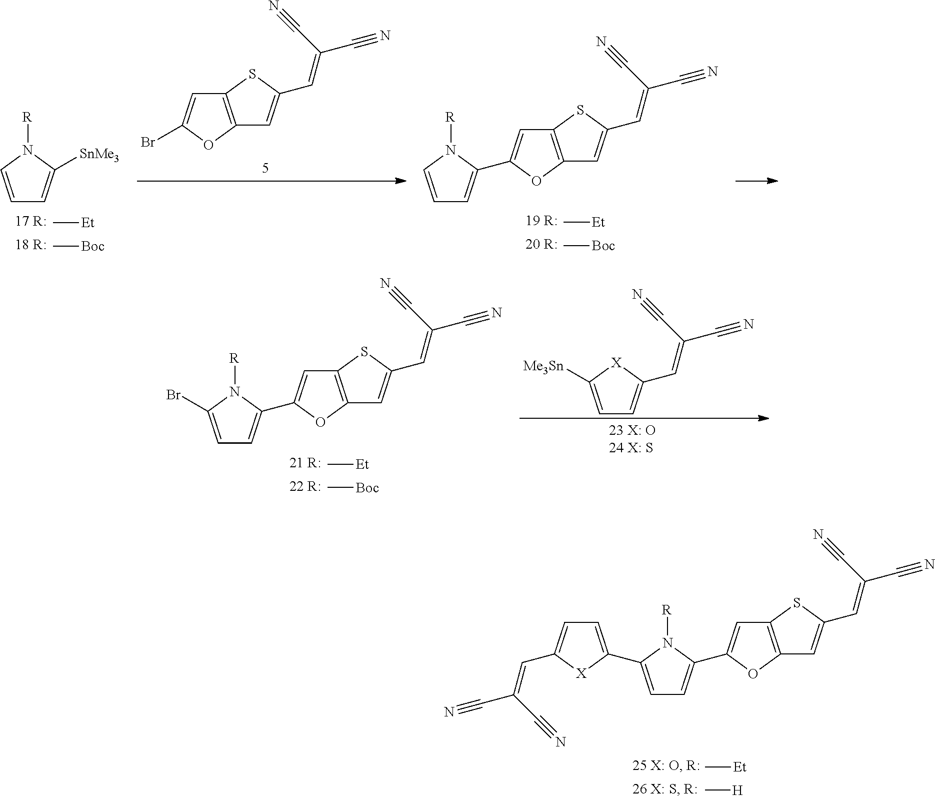

Compound 17 can be synthesized according to tetrahedron, 71 (33), 5399-5406; 2015. Compound 18 can be synthesized according to Chemistry-A European Journal, 21 (5), 2003-2010; 2015. Compounds 23 and 24 can be used according to Eur. Pat. Appl., 3187496, 2017 can be synthesized.

### Synthesis of {[2-(1-ethyl-1H-pyrrol-2-yl) thieno[3,2-b]furan-5-yl]methylidene}propanedinitriles (19)

910 mg (3.53 mmol) of 1-ethyl-2-(trimethylstannyl) 1H-pyrrole 17 and mmol) 820 mg (2.94 of [(2-bromothieno[3,2-b]furan-5-yl)methylidene]propanedinitriles 5 was dissolved in 15 ml of dioxane, and the solution was degassed. 37.6 mg (0.0735 mmol) of bis(tri-tert.-butylphosphine) palladium (0) was added, and the reaction mixture was stirred at 80° C. overnight. After the reaction mixture had cooled to room temperature, the precipitate was filtered off and washed with methanol. The crude product was purified by column chromatography on silica gel in dichloromethane, 439 mg (51% yield) was isolated from {[2-(1-ethyl-1H-pyrrol-2-yl) thieno[3,2-b]furan-5-yl]methylidene}propanedinitrile 19. 1H-NMR in d6-dimethylsulfoxide at 90° C.; ppm: 8.49 (s, 1H), 7.97 (s, 1H), 7.19 (s, 1H), 7.10 (dd, 1H), 6.74 (dd, 1H), 6.21 (dd, 1H), 4.23 (q, 2H), 1.36 (t, 3H). Compound 20 was synthesized analogously from compound 18.

### Synthesis of 2-bromo-5-[5-(2,2-dicyanoethenyl) thieno[3,2-b]furan-2-yl]-1H-pyrrol-1-yl (21)

513 mg (1.75 mmol) of {[2-(1-ethyl-1H-pyrrol-2-yl) thieno[3,2-b]furan-5-yl]methylidene}propanedinitrile 19 was dissolved in 30 ml DMF under argon. A solution of 280 mg (1.57 mmol) of NBS in 5 ml DMF was added to this solution at −78° C., and the reaction mixture was stirred overnight at −18° C. The reaction mixture was placed on ice, and dichloromethane was added. The aqueous phase was extracted with dichloromethane. The crude product was recrystallized from ethanol, 510 mg (78% yield) was isolated from 2-bromo-5-[5-(2,2-dicyanoethenyl) thieno[3,2-b]furan-2-yl]-1H-pyrrol-1-yl 21. 1H-NMR in d6-acetone, ppm: 8.37 (s, 1H), 7.98 (s, 1H), 7.25 (s, 1H), 6.81 (d, 1H), 6.36 (d, 1H), 4.39 (q, 2H), 1.4 (t, 3H). Compound 22 was synthesized analogously from compound 20.

### Synthesis of [(2-{5-[5-(2,2-dicyanoethenyl) furan-2-yl]-1-ethyl-1H-pyrrol-2-yl}thieno[3,2-b]furan-5-yl)methylidene]propanedinitrile (A25)

255 mg (0.685 mmol) of 2-bromo-5-[5-(2,2-dicyanoethenyl) thieno[3,2-b]furan-2-yl]-1H-pyrrol-1-yl 21 and 252 mg (0.822 mmol) of 5-(trimethylstannyl)-2-dicyanovinylfuran 23 was dissolved in 3.5 ml of dioxane and it was degassed. To the reaction mixture was added 8.75 mg (0.017 mmol) of bis-(tri-tert-butylphosphine)-palladium, and it was stirred overnight at 60° C. After the reaction mixture had cooled to room temperature, the precipitate was filtered off. The crude product was washed with methanol and recrystallized from toluene and chlorobenzene. Subsequently, the product was sublimated, with 114 mg (yield 38%, HPLC purity 99.7% @542 nm) being isolated from [(2-{5-[5-(2,2-dicyanoethenyl) furan-2-yl]-1-ethyl-1H-pyrrol-2-yl}thieno[3,2-b]furan-5-yl)methylidene]propanedinitrile A25. Compound A26 was synthesized analogously from compounds 22 and 24.

### Synthesis of compound A37 and compound A38

### Synthesis of {[3-chloro-5-(5-{5-[5-(2,2-dicyanoethenyl) thieno[3,2-b]furan-2-yl]-1-ethyl-1H-pyrrol-2-yl}furan-2-yl)thiophen-2-yl]methylidene}propanedinitrile (A37) and {[2-(5-{5-[5-(2,2-dicyanoethenyl)-1,3-thiazol-2-yl]furan-2-yl}-1-ethyl-1H-pyrrol-2-yl) thieno[3,2-b]furan-5-yl]methylidene}propanedinitriles (A38)

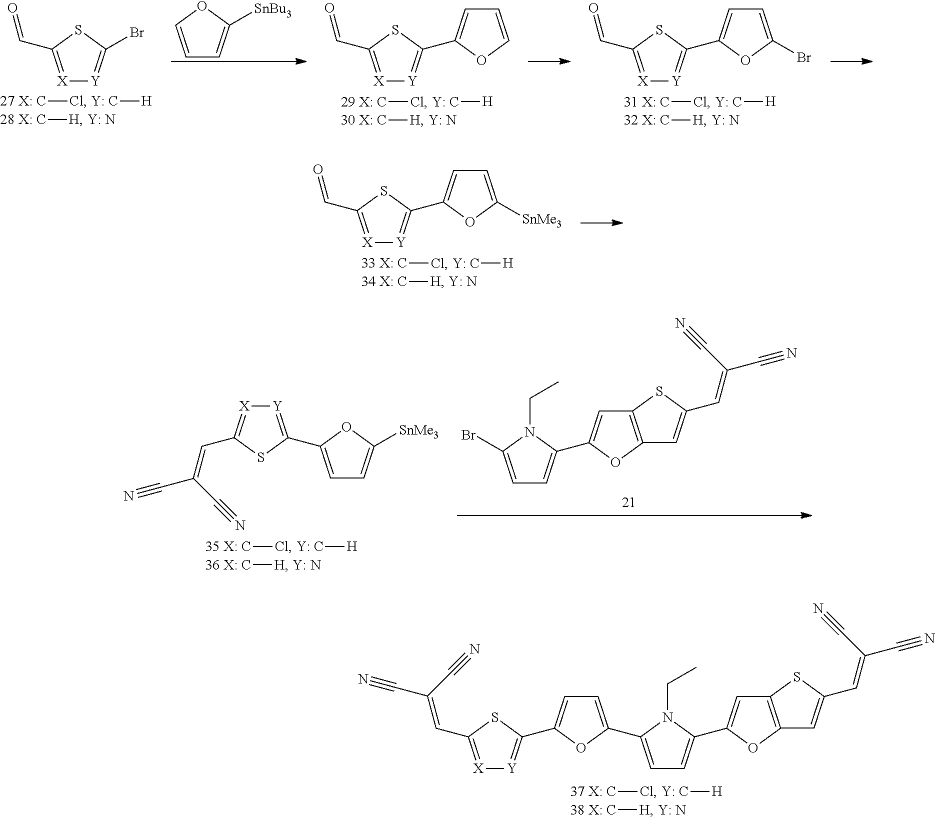

### Synthesis of 3-chloro-5-(furan-2-yl)thiophene-2-carbaldehyde (29)

7.27 g (19.8 mmol) of 2-(tributhylstannyl) furan was dissolved in 99 ml of dioxane. 5 g (21.7 mmol) of 5-bromo-3-chlorothiophene-2-carbaldehyde was added, and the solution was degassed. 252 mg (0.49 mmol) of bis-(tri-tert-butylphosphine)-palladium (0) was added to the reaction mixture and stirred for 4 h at 60° C. After the reaction mixture had cooled to room temperature, dichloromethane was added. The organic phase was washed with water and sat. NaCl solution. The crude product was purified by silica gel filtration in dichloromethane, and 10.8 g (110% yield, purity 43%) was isolated from 3-chloro-5-(furan-2-yl)thiophene-2-carbaldehyde 29. 1H-NMR in d6-acetone, ppm: 10.01 (s, 1H), 7.77 (dd, 1H), 7.48 (s, 1H), 7.12 (dd, 1H), 6.67 (dd, 1H). Compound 30 can be synthesized analogously from 2-bromo-1,3-thiazole-5-carbaldehydes.

### Synthesis of 5-(5-bromofuran-2-yl)-3-chlorothiophene-2-carbaldehyde (31)

10.8 g (21.8 mmol) of 3-chloro-5-(furan-2-yl)thiophene-2-carbaldehyde 29 was dissolved in 50 ml of N, N-dimethylformamide. 3.96 g (21.8 mmol) of N-bromosuccinimide was added in small portions at 0° C., and the reaction mixture was stirred overnight at room temperature. The reaction mixture was placed on ice; the precipitate was filtered and washed with water. The crude product was dried in air and purified by column chromatography on silica gel in 50% petroleum ether in dichloromethane. In the process, 4.36 g (69% yield) was cleanly isolated from 5-(5-bromofuran-2-yl)-3-chlorothiophene-2-carbaldehyde 31. 1H-NMR in d6-acetone, ppm: 10.02 (s, 1h), 7.51 (s, 1H), 7.15 (d, 1H), 6.73 (d, 1H). Compound 32 can be synthesized analogously.

### Synthesis of 3-chloro-5-[5-(trimethylstannyl) furan-2-yl]thiophene-2-carbaldehyde (33)

1.8 ml (16.4 mmol) of 1-methylpiperazine was dissolved in 75 ml of tetrahydrofuran. At −78° C., 6.5 ml (16.4 mmol) of 2.5 N n-buthyllithium was added dropwise. 4.34 g (14.9 mmol) of 5-(5-bromofuran-2-yl)-3-chlorothiophene-2-carbaldehyde 31 was dissolved in 64 ml of tetrahydrofuran and added dropwise to the reaction solution at −78° C. 2.73 ml (17.9 mmol) of N, N,N′,N′-tetramethylethylenediamine was added to the reaction solution. Subsequently, 7.15 ml (19.9 mmol) of 2.5 N n-buthyllithium was added to the reaction mixture and stirred for 1 h at −78° C. 17.9 ml (17.9 mmol) of trimethylstannyl chloride was added to the reaction mixture, and it was allowed to warm to room temperature overnight. Sat. NaCl solution and methyl tert-butyl ether were added to the reaction mixture. The organic phase was washed with water and sat. NaCl solution. The crude product 5.5 g (98.3% yield) was isolated from 3-chloro-5-[5-(trimethylstannyl) furan-2-yl]thiophene-2-carbaldehyde 33 and reacted without purification.

Compound 34 can be synthesized analogously.

### Synthesis of ({3-chloro-5-[5-(trimethylstannyl) furan-2-yl]thiophen-2-yl}methylidene) propanedinitrile (35)

5.48 g (14.6 mmol) of 3-chloro-5-[5-(trimethylstannyl) furan-2-yl]thiophene-2-carbaldehyde 33 was dissolved in 14 ml of ethanol. 1.16 mg (17.5 mmol) of malodinitrile and 119 mg (1.31 mmol) β-alanine was added, and the reaction mixture was stirred overnight at room temperature. The precipitate was filtered off and washed with ethanol, wherein 2.1 g (34% yield) was isolated from ({3-chloro-5-[5-(trimethylstannyl) furan-2-yl]thiophen-2-yl}methylidene) propanedinitrile A35, which was reacted without further purification. 1H-NMR in d6-acetone, ppm: 8.30 (s, 1H), 7.63 (s, 1H), 7.26 (d, 1H), 6.91 (d, 1H), 0.41 (s, 9H). Compound 36 can be synthesized analogously.

### Synthesis of {[3-chloro-5-(5-{5-[5-(2,2-dicyanoethenyl) thieno[3,2-b]furan-2-yl]-1-ethyl-1H-pyrrol-2-yl}furan-2-yl)thiophen-2-yl]methylidene}propanedinitrile (A37)

457 mg (1.23 mmol) {[2-(5-bromo-1-ethyl-1H-pyrrol-2-yl) thieno[3,2-b]furan-5-yl]methylidene}propanedinitriles 21 and 624 mg (1.47 mmol) ({3-chloro-5-[5-(trimethylstannyl) furan-2-yl]thiophene-2-yl}methylidene) propanedinitriles 35 was dissolved in 2.5 ml dioxane and degassed. 15.7 mg (0.03 mmol) of bis-(tri-tert-butylphosphine)-palladium (0) was added, and the reaction mixture was stirred overnight at 60° C. After the reaction mixture had cooled to room temperature, the precipitate was filtered and washed with methanol. The crude product was recrystallized from toluene and sublimated, wherein 37 mg (5.5% yield, HPLC purity 98.0% @ 567 nm) was isolated from {[3-chloro-5-(5-{5-[5-(2,2-dicyanoethenyl) thieno[3,2-b]furan-2-yl]-1-ethyl-1H-pyrrol-2-yl}furan-2-yl)thiophen-2-yl]methylidene}propanedinitrile A37. Compound 38 can be synthesized analogously.

1. Compound of the general formula I
A1-M-(T1)ₐ-(T2)_{b}-(T3)_{c}-A2 (I)
with the parameters a, b, c in each case independently of one another 0, 1 or 2, with the proviso that at least one of the parameters a, b, c=1 or 2,
with group M selected from the group consisting of: 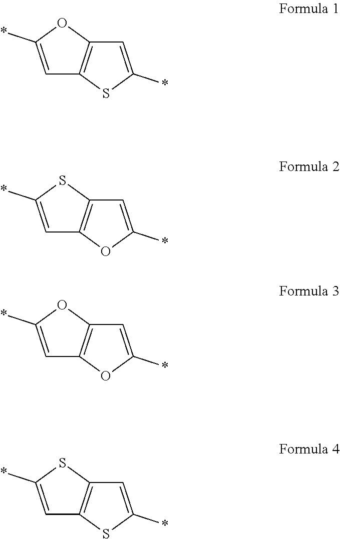 [SMILES: *c1cc2sc(*)cc2o1 ; *c1cc2sc(*)cc2o1 ; *c1cc2oc(*)cc2o1 ; *c1cc2sc(*)cc2s1]
wherein * denotes the link to the groups T1, T2, T3, A1 and A2, wherein H atoms of Formulas 1 to 4 may be substituted by alkyl, O-alkyl, S-alkyl or halogen;
with the groups T1, T2 and T3 independently selected from the group consisting of: 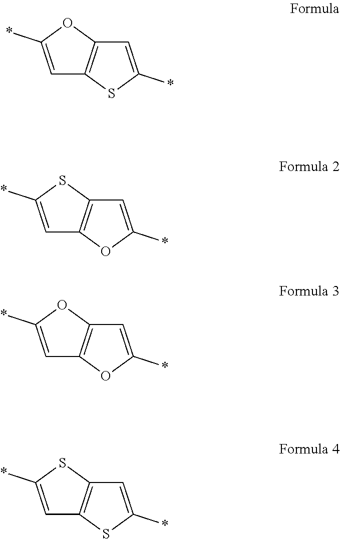 [SMILES: *c1cc2sc(*)cc2o1 ; *c1cc2sc(*)cc2o1 ; *c1cc2oc(*)cc2o1 ; *c1cc2sc(*)cc2s1]
wherein H atoms of Formulas 1 to 4 may be substituted by alkyl, O-alkyl, S-alkyl or halogen, 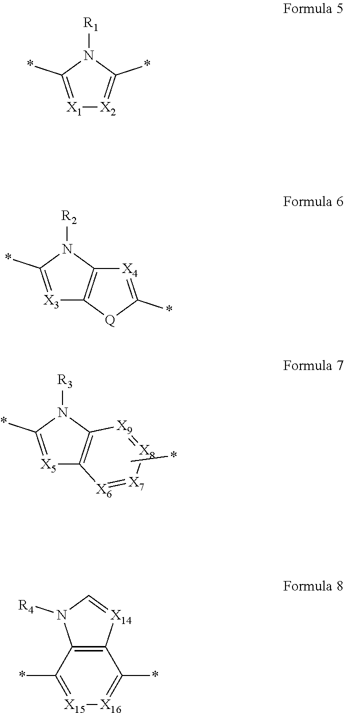
with R₁ to R₄ each independently selected from the group consisting of H, alkyl, alkenyl, aryl and heteroaryl; 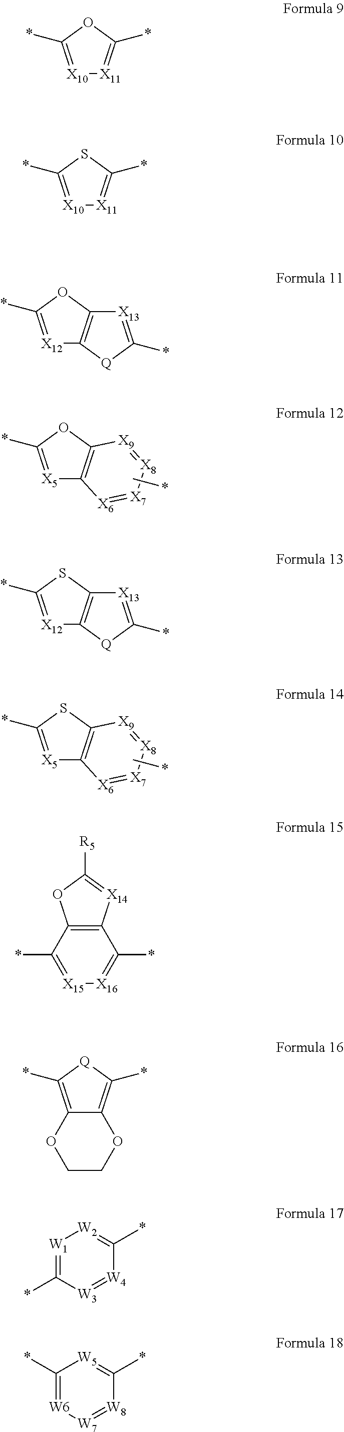
with X₁ to X₁₆, selected independently from N or CR₆, with the proviso that, in the Formulas 7, 12 and 14, a group of X₆ to X₉ is denoted by the link * to a further group in the compound of the general Formula I,
with Q selected from the group consisting of O, S, and NR₇,
with W₁ to W₈ each independently selected from N and CR₈,
wherein R₆, R₇ and R₈ are each independently selected from the group consisting of H, CN, alkyl, alkenyl, O-alkyl, S-alkyl, aryl, and heteroaryl;
wherein the electron-attracting groups A1 and A2 are, independently of one another, electron-attracting groups having at least one C═C double bond, and wherein * denotes the link to a further group M, T1, T2, T3 and A2 in the compound of the general formula I.

2. Compound of claim 1, wherein T1 is independently selected from the group consisting of: 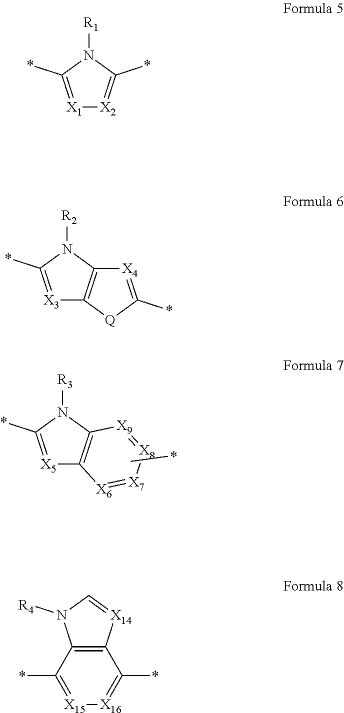
wherein preferably X₁ and X₂ and/or X₃ and X₄ are CR₆, preferably R₆ is selected from H and alkyl; and/or wherein preferably X₅ is CR₆ with R₆ H or alkyl, and/or wherein preferably X₆ to X₉ is CR₆, and/or wherein preferably R₁ to R₃ are, independently of one another, H, alkyl or aryl, and/or wherein preferably R₄ is H or alkyl.

3. Compound according to claim 1 or 2, wherein T2 and T3 are selected from the group consisting of: 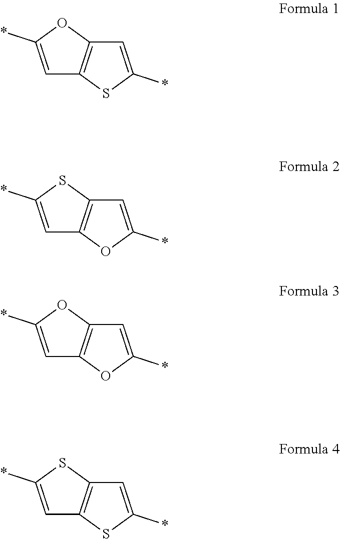 [SMILES: *c1cc2sc(*)cc2o1 ; *c1cc2sc(*)cc2o1 ; *c1cc2oc(*)cc2o1 ; *c1cc2sc(*)cc2s1]
wherein H atoms of Formulas 1 to 4 may be substituted by alkyl, O-alkyl, S-alkyl or halogen; preferably H atoms of Formulas 1 to 4 are not substituted, 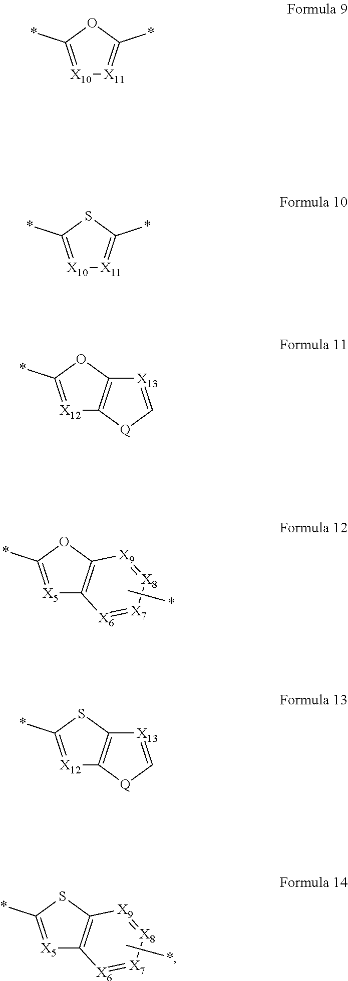
wherein preferably R₆ and R₇ are each independently selected from the group consisting of H, alkyl, and aryl.

4. Compound according to one of the preceding claims, wherein A1 is selected from the group consisting of: 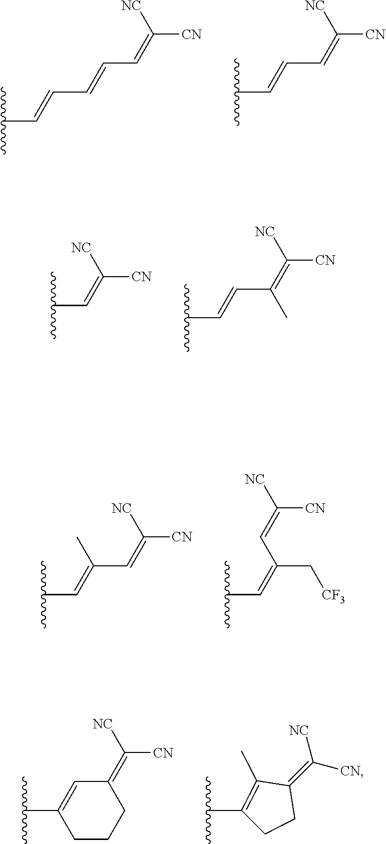
and
wherein A2 is selected from the group consisting of:

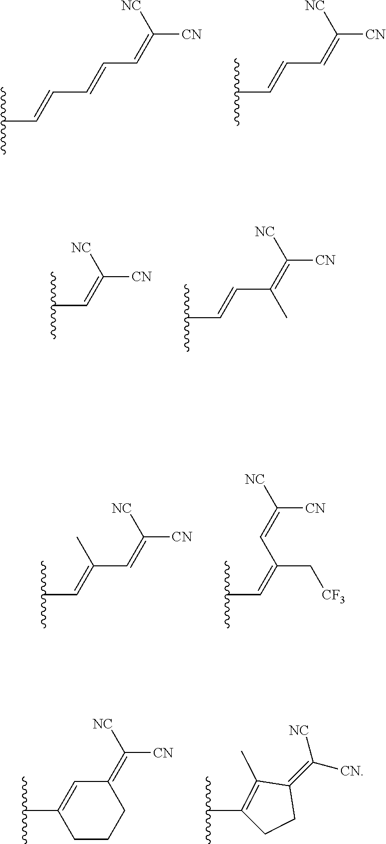

5. Compound according to one of the preceding claims, wherein the parameters a, b, c are preferably each, independently of one another, 0 or 1, with the proviso that at least one of the parameters a, b, c is equal to 1, preferably at least two of the parameters a, b, c are equal to 1; or wherein the parameter a is equal to 1 or 2, with at least one of the parameters b and c being equal to 1 or 2, preferably with one of the parameters b or c being equal to 1 or 2, preferably with one of the parameters b or c being equal to 1, more preferably with the parameter c being equal to 1 or 2.

6. Compound according to one of the preceding claims,
wherein the group M is selected from the group consisting of: 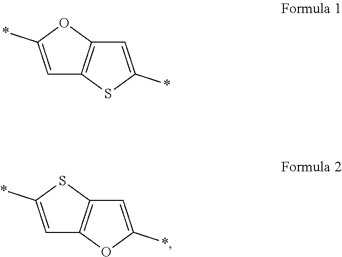
and
wherein T1 is selected from the group consisting of 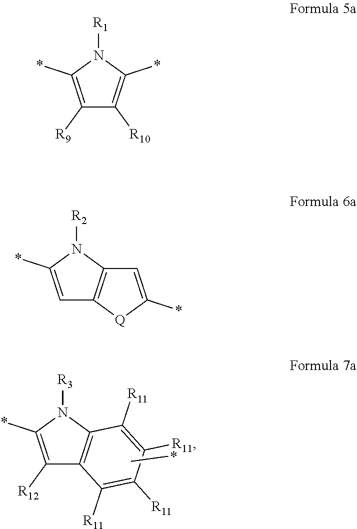
wherein R₉ and R₁₀ are each independently selected from the group consisting of H, halogen, preferably F and Cl, CN, alkyl, alkenyl, O-alkyl, S-alkyl, aryl, heteroaryl; preferably R₉ and R₁₀ are independently H or alkyl,
wherein R₁₁ and R₁₂ are each independently selected from the group consisting of H, halogen, preferably F and Cl, CN, alkyl, alkenyl, O-alkyl, S-alkyl, aryl, heteroaryl; preferably R₁₁ and R₁₂ are H.

7. Compound according to one of the preceding claims, wherein the compound has the general formula II
A1-M-(T1)ₐ-(T3)_{c}-A2 (II),
wherein preferably a and c are equal to 1, a is equal to 1 and c is equal to 2, or a is equal to 2 and c is equal to 1.

8. Compound according to one of the preceding claims, wherein the compound is selected from the group consisting of:

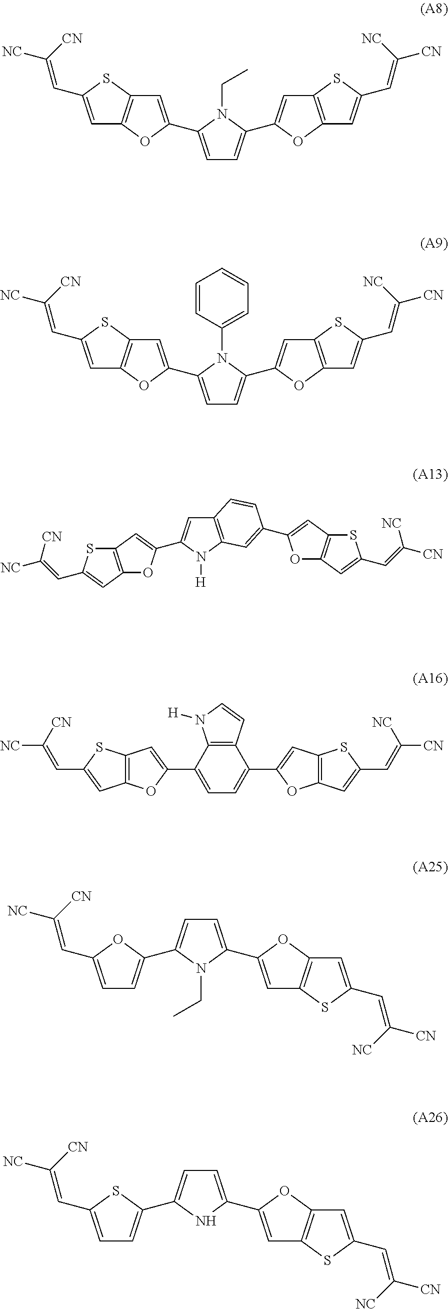 [SMILES: CCn1c(-c2cc3sc(C=C(C#N)C#N)cc3o2)ccc1-c1cc2sc(C=C(C#N)C#N)cc2o1 ; N#CC(C#N)=Cc1cc2oc(-c3ccc(-c4cc5sc(C=C(C#N)C#N)cc5o4)n3-c3ccccc3)cc2s1 ; N#CC(C#N)=Cc1cc2oc(-c3ccc4cc(-c5cc6sc(C=C(C#N)C#N)cc6o5)[nH]c4c3)cc2s1 ; N#CC(C#N)=Cc1cc2oc(-c3ccc(-c4cc5sc(C=C(C#N)C#N)cc5o4)c4[nH]ccc34)cc2s1 ; CCn1c(-c2ccc(C=C(C#N)C#N)o2)ccc1-c1cc2sc(C=C(C#N)C#N)cc2o1 ; N#CC(C#N)=Cc1ccc(-c2ccc(-c3cc4sc(C=C(C#N)C#N)cc4o3)[nH]2)s1]

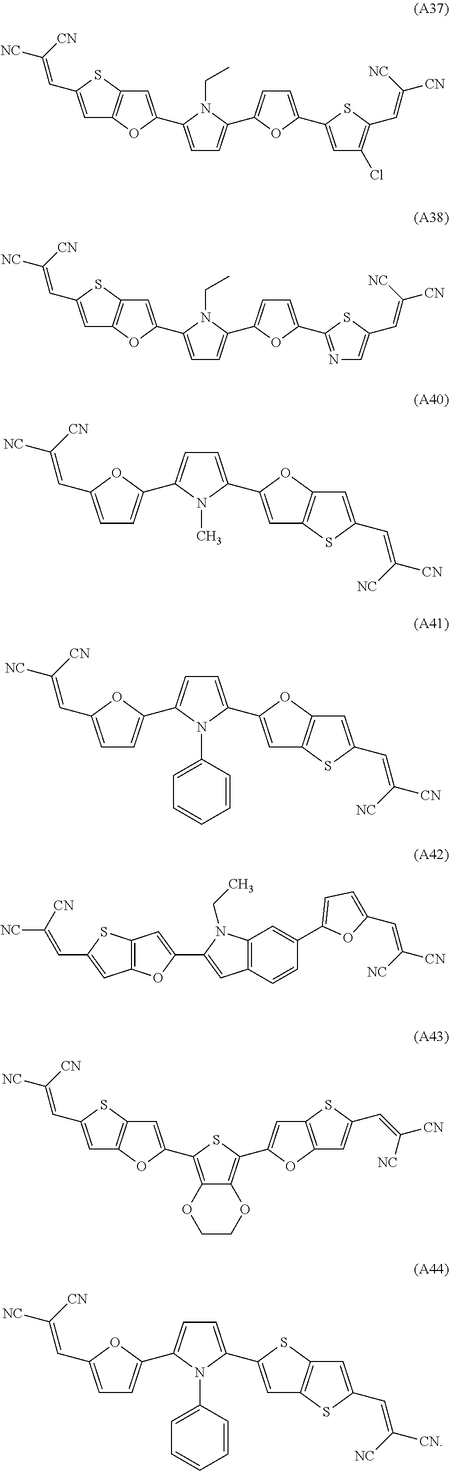

9. Use of at least a compound according to one of claims 1 through 8 in an organic electronic device, preferably in an organic photovoltaic element.

10. Organic electronic device comprising an electrode, a counter-electrode and at least one organic photoactive layer between the electrode and the counter-electrode, wherein the at least one organic photoactive layer has at least one compound according to one of claims 1 through 8.
